(19) United States
(12) Patent Application Publication
(10) Pub. No.: US 2011/0158913 A1
(43) Pub. Date: Jun. 30, 2011

(54) Antisense and pretargeting optical imaging

(75) Inventors: Don Hnatowich, Brookline, MA (US); Kayoko Nakamura, Tokyo (JP); Yi Wang, Worcester, MA (US); Xinrong Liu, Worcester, MA (US); Jiang He, San Mateo, CA (US); Surong Zhang, Shrewsbury, MA (US); Mary Rusckowski, Southborough, MA (US)

(73) Assignee: University of Massachusetts, Boston, MA (US)

(21) Appl. No.: 12/009,452

(22) Filed: Jan. 18, 2008

Related U.S. Application Data
(60) Provisional application No. 60/881,406, filed on Jan. 19, 2007.

(51) Int. Cl. A61K 49/00

(52) U.S. Cl. 424/9.6; 424/9.1

(57) ABSTRACT

The present invention relates, in part, to detectably labeled oligomer duplexes and their use in optical imaging, including, in vivo optical imaging. The invention includes methods of optical imaging including in vivo pretargeting methods and in vivo antisense optical imaging methods.

### RELATED APPLICATIONS

This application claims the benefit under 35 U.S.C. §119(e) of U.S. provisional application Ser. No. 60/881,406, filed Jan. 19, 2007, the disclosure of which is incorporated by reference herein in its entirety.

### GOVERNMENT SUPPORT

This invention was made in part with government support under grant number RO1 CA94994 from the National Institutes of Health (NIH) and DE-FG02-03ER63602 from the Department of Energy. The U.S. government has certain rights in this invention.

### FIELD OF THE INVENTION

The invention relates in part to the use of detectably labeled oligomer duplexes for optical imaging, including, in vivo optical imaging.

### BACKGROUND OF THE INVENTION

An alternative technique to imaging using radioactivity that is gaining in prominence is optical (fluorescence and bioluminescence) imaging. Optical imaging can be used for surface imaging in patients with skin cancers, in exposed tissues, and in tissues accessible by endoscopy (Chen, C. S. et al., Br J Dermatol 153: 1031-1036, 2005). Optical and radioactivity imaging share the potential for both high resolution and high sensitivity imaging (Houston, J. P. et al., J Biomed Opt 10: 054010, 2005; Bloch, S. et al., J Biomed Opt 10: 054003, 2005).

Recent studies have investigated antisense DNAs and other oligomers and the use of double-stranded duplexes to improve cellular delivery (Liu, X. et al., Mol Imaging Biol 2006. Improved delivery in cell culture of radio-labeled antisense DNAs by duplex formation (Epub ahead of print)). Single-stranded and double-stranded DNAs or siRNA have been compared and double-stranded oligomers (Liu, X. et al., Mol Imaging Biol 2006. Improved delivery in cell culture of radio-labeled antisense DNAs by duplex formation (Epub ahead of print); Jekerle, V. et al., J Pharm Sci 8: 516-527, 2005; Asriab-Fisher, A. et al., Biochem Pharmacol 68: 403-407, 2004) are stable both in cell culture and in animal studies (Aharinejad, S. et al., Cancer Res 64: 5378-5384, 2004) and appear to show greater accumulation in cells compared to single strand oligomers (Liu, X. et al., Mol Imaging Biol 2006. Improved delivery in cell culture of radio-labeled antisense DNAs by duplex formation (Epub ahead of print)).

### SUMMARY OF THE INVENTION

According to one aspect of the invention, methods of optically imaging a target entity in a subject are provided. The methods include (a) administering to the subject a binding olecule that comprises an oligomer moiety and specifically binds to the target entity, (b) contacting the binding molecule with a quenched first linear oligomer duplex comprising a detectably labeled oligomer, wherein the detectably labeled oligomer is hybridized to a quenching oligomer; the detectable label is quenched unless the detectably labeled oligomer and the quenching oligomer dissociate; and the detectably labeled oligomer has a higher affinity to form a linear duplex with the oligomer moiety of the binding molecule than to form a linear duplex with the quenching oligomer, and (c) detecting the presence of unquenched detectable label in the subject, wherein the presence of unquenched detectable label indicates that the detectably labeled oligomer has dissociated from the quenching oligomer and hybridized to the oligomer of the binding molecule to form a second linear oligomer duplex, permitting optical imaging of the target entity in the subject. In some embodiments, one or more of the oligomers are phosphodiester, phosphorothioate, peptide nucleic acid (PNA), locked nucleic acid (LNA), and/or phosphorodiamidate morpholino (MORF) oligomers. In some embodiments, the detectable label is a fluorescent or bioluminescent label. In certain embodiments, the fluorescent label is a Cy5.5 emitter. In some embodiments, the quenching oligomer comprises a quenching moiety. In some embodiments, the quenching moiety is BHQ3 or Iowa black. In some embodiments, the detectable label is detected in vivo. In certain embodiments, the detectable label is detected in real time. In some embodiments, the first linear oligomer duplex comprises two oligomers that are not both phosphodiester, phosphorothioate, peptide nucleic acid (PNA), locked nucleic acid (LNA), or phosphorodiamidate morpholino (MORF) oligomers. In some embodiments, the oligomer moiety is a single-stranded nucleic acid moiety. In some embodiments, the binding molecule is an antibody or antigen-binding fragment thereof. In some embodiments, the target entity is a polypeptide, nucleic acid, polysaccharide or lipid molecule. In certain embodiments, the target entity is a cell. In some embodiments, the cell is a cancer cell. In some embodiments, the subject is human. In some embodiments, presence of the specific target entity is associated with a disease or disorder. In certain embodiments, the disease is cancer. In some embodiments, the optical imaging of the target entity in the subject is diagnostic for a disease or disorder in the subject. In some embodiments, the disease is cancer.

According to yet another aspect of the invention, methods of optically imaging a target entity in a subject are provided. The methods include (a) administering to a subject a binding molecule that specifically binds to the target entity and comprises a partially hybridized oligomer duplex with a first fluorescent label, (b) contacting the binding molecule with an oligomer comprising a second fluorescent label, wherein the oligomer specifically hybridizes to the unhybridized region of the oligomer duplex, and the hybridization of the oligomer with the oligomer duplex results in a shift in the fluorescence frequency of at least one of the fluorescent labels, and (c) detecting the presence of the shift in fluorescence frequency in the subject, wherein the shift in fluorescence frequency in the subject indicates hybridization of the oligomer to the oligomer duplex, permitting optical imaging of the target entity in the subject. In some embodiments, one or more of the oligomers are phosphodiester, phosphorothioate, peptide nucleic acid (PNA), locked nucleic acid (LNA), and/or phosphorodiamidate morpholino (MORF) oligomers. In certain embodiments, the first and second fluorescent labels are fluorescent resonance energy transfer (FRET) pairs or are bioluminescent resonance energy transfer (BRET) pairs. In some embodiments, the detectable label is detected in vivo. In some embodiments, the detectable label is detected in real time. In some embodiments, the binding molecule is an antibody or antigen-binding fragment thereof. In certain embodiments, the target entity is a polypeptide, nucleic acid, polysaccharide, or lipid molecule. In some embodiments, the target entity is a cell. In some embodiments, the cell is a cancer cell. In certain embodiments, presence of the specific target entity is associated with a disease or disorder. In some embodiments, the disease is cancer. In some embodiments, the subject is human. In certain embodiments, the optical imaging of the target entity in the subject is diagnostic for a disease or disorder in the subject. In some embodiments, the disease is cancer.

According to yet another aspect of the invention, methods of optically imaging a target entity in a subject are provided. The methods include (a) administering to the subject a linear oligomer duplex comprising a detectably labeled oligomer and a quenching oligomer, wherein the detectable label is quenched by the quenching oligomer unless the detectably labeled oligomer and quenching oligomer of the duplex dissociate, and wherein the detectably labeled oligomer has a higher affinity to form a duplex with a specific target nucleic acid than to form a duplex with the quenching oligomer, and (b) detecting the presence of unquenched detectable label in the subject, wherein the presence of unquenched detectable label in the subject indicates that the detectably labeled oligomer has formed a duplex with the specific target nucleic acid, permitting optical imaging of the target nucleic acid in the subject. In some embodiments, one or more of the oligomers are phosphodiester, phosphorothioate, peptide nucleic acid (PNA), locked nucleic acid (LNA), and/or phosphorodiamidate morpholino (MORF) oligomers. In some embodiments, the detectable label is a fluorescent or bioluminescent label. In certain embodiments, the fluorescent label is a Cy5.5 emitter. In some embodiments, the quenching oligomer comprises a quenching moiety. In some embodiments, the quenching moiety is BHQ3 or Iowa black. In certain embodiments, the detectable label is detected in vivo. In some embodiments, the detectable label is detected in real time. In some embodiments, the specific target nucleic acid is an mRNA. In some embodiments, the detectably labeled oligomer is an antisense sequence to the specific target nucleic acid. In certain embodiments, expression of the specific target nucleic acid is associated with a disease or disorder. In some embodiments, the disease is cancer. In certain embodiments, the subject is human. In some embodiments, the optical imaging of the target nucleic acid in the subject is diagnostic for a disease or disorder in the subject. In some embodiments, the disease is cancer. In some embodiments, the detectably labeled oligomer and the quenching oligomer are not both phosphodiester, phosphorothioate, peptide nucleic acid (PNA), locked nucleic acid (LNA), or phosphorodiamidate morpholino (MORF) oligomers. In certain embodiments,

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

FIG. 1 shows a Surface Plasmon Resonance (SPR) sensogram depicting the association (region A) of the MORF25 (MORF=morpholino) to the immobilized 18 mer complementary DNA (cDNA18) to form the heteroduplex and its slow dissociation (region B), and showing the dissociation of the MORF25/cDNA18 heteroduplex with the formation and release of the MORF25/cMORF25 homoduplex following two injections of the cMORF25 (regions C and D) to the immobilized heteroduplex. The figure shows that the immobilized cDNA18 was able to capture the MORF25 but unable to retain it in the presence of cMORF25.

FIG. 2 shows a digitalized image of a whole body fluorescence image of SKH mice implanted subcutaneously with microspheres with (right thigh) or without (left thigh) cMORF25. FIG. 2A shows imaging at 5 min and FIG. 2B shows imaging at 60 min post administration of the Cy5.5-MORF25/BHQ3-cDNA18 duplex (left mouse) or Cy5.5-MORF25 singlet (right mouse).

FIG. 3 provides a graph of the fluorescent intensities of the target thigh with cMORF25 microspheres and the control thigh with native microspheres over time in animals receiving the Cy5.5-MORF25/BHQ3-cDNA18 duplex or the Cy5.5-MORF25 singlet. Fluorescent intensity in p/s/cm²/sr. Error bars represent one SD of the mean (N=3).

FIG. 4 shows a graph of the relative fluorescence intensities of the control singlet PS DNA25-Cy5.5 (closed circles) and the study duplex PS DNA25-Cy5.5/PO cDNA18-BHQ3 (open circles). FIG. 4A shows relative fluorescent intensities at 37° C. 10% FBS/DMEM over 24 h. FIG. 4B shows relative fluorescent intensities in 70% normal mouse serum over 24 h. Error bars represent one SD of the mean (N=3).

FIG. 5 shows graphs of the flow cytometry of KB-31 cells and KB-G2 cells. FIG. 5A shows KB-31 cells before incubation with the study duplex. FIG. 5B shows KB-31 cells after incubation with the study duplex. FIG. 5C shows KB-G2 cells before incubation with the study duplex. FIG. 5D shows KB-G2 cells after incubation with the study duplex.

FIG. 6 shows graphs of the relative cellular fluorescence intensity. FIG. 6A shows relative cellular fluorescence intensity at 3 h in KB-G2 cells after incubation with the DNA25-Cy5.5 singlet or the study DNA25-Cy5.5/cDNA18-BHQ3 duplex at three dosages. FIG. 6B shows the relative cellular fluorescence intensity at 3 h in KB-31 and KB-G2 cells after incubation with 0.1 nmol of the study or control duplex. Error bars represent one SD of the mean (N=3). The higher fluorescence of KB-G2 cells when incubated with the study duplex and the identical florescence when incubated with the control duplex are both evidence of specific binding.

FIG. 7 shows digitized fluorescence images of KB-G2 cells. FIG. 7A shows images of live KB-G2 cells incubated with the DNA25-Cy5.5 as the singlet. FIG. 7B shows images of live KB-G2 cells incubated with the DNA25-Cy5.5 as the duplex. FIG. 7C-E show fluorescence images of fixed KB-G2 cells previously incubated with the DNA25-Cy5.5 as the singlet. FIG. 7F-H show fluorescence images of fixed KB-G2 cells previously incubated with the DNA25-Cy5.5 as the duplex. The cells were incubated with the DNAs at 0.3 μM for 3 h at 37° C. Nuclei were labeled with Sytox Green (Magnification×400).

FIG. 8. shows whole body fluorescent images of KB-G2 tumor bearing mice in the dorsal view at 5 h following administration of 3 nmol of the study DNA25-Cy5.5/cDNA18-BHQ3 duplex or control cDNA18-Cy5.5/DNA25-BHQ3 duplex. FIG. 8A shows an image of tumor bearing mice following administration of the study DNA25-Cy5.5/cDNA18-BHQ3 duplex. FIG. 8B shows an image of tumor bearing mice following administration of the control cDNA18-Cy5.5/DNA25-BHQ3 duplex. Also presented are microscopic analysis of tissue sections of tumor obtained at 24 h after injection of both study and control duplexes. The arrow indicates the tumor location (Magnification ×100). FIG. 8C, FIG. 8D and FIG. 8E show GFP, Cy5.5, and overlap of the two fluorescent signals for the study duplex, respectively. FIGS. 8F, FIG. 8G, and FIG. 8H show GFP, Cy5.5 and overlap of the two fluorescent signals for the study duplex, respectively.

FIG. 9 shows the graph of a tumor-to-normal thigh fluorescent ratio at 0.5, 3, 5 and 24 h post administration in one animal receiving the control duplex and in one study animals receiving either 1, 3 or 5 nmoles of study duplex. The histograms all show higher fluorescence in the tumored thigh in the study animals compared to the control animal.

FIG. 10 is a schematic diagram of a kit for optical imaging (10=kit, 12=component for optical imaging; 14=additional components; 20=instructions).

FIG. 11 presents histograms showing fluorescence intensity. FIG. 11A shows fluorescence intensity of wells containing the study PS Cy5.5 cDNA1 (open bars) and control PS Cy5.5 rDNA (closed bars) 30 min after the addition of PS Iowa Black DNA1 at different DNA1:cDNA1 molar ratios from 0:1 to 3:1 in PBS buffer. Error bars represent one SD (N=3). FIG. 11B shows fluorescence intensities of wells with MORF1/PO Cy5.5 cc′DNA microspheres at 20 min (open bars) and 60 min (hatched bars) following addition of PS Iowa Black DNA2 at 0 to 15 fold molar excess to PO Cy5.5 cc′DNA in PBS/BSA binding buffer. Error bars represent one SD (N=3).

FIG. 12 presents histograms showing quenching efficiency. FIG. 12A shows the quenching efficiency in SKH-1 mice receiving iv 0.8 nmol of PS Cy5.5 cDNA1 followed by a 3-fold molar excess of PS Iowa Black DNA1 10 min later. Results presented for both the dorsal view (open bars) and ventral view (hatched bars) obtained between 20 and 120 min thereafter. Error bars represent one SD (N=3). FIG. 12B shows quenching efficiency 10 to 90 min after iv administration of 6.0 nmol of PS Iowa Black DNA2 in SKH-1 mice implanted with 0.06 nmol PO Cy5.5 cc′DNA microspheres. Results presented for both dorsal view (open bars) and ventral view (hatched bars). Error bars represent one SD (N=4).

### DETAILED DESCRIPTION

Imaging can be performed using different types of detectable labels. Examples of different categories of imaging are: imaging using radionuclides and imaging using optical agents. Although radionuclides and other radioactive agents are very powerful imaging agents, they are not easily manipulated. In contrast, optical imaging allows for fine-tuning of administration and detection regimens including separation of the moment of targeting and the moment of imaging. Pre-targeted optical imaging methods of the invention permit enhanced optical imaging to be performed for improved imaging in cells, tissues, and subjects. The invention, in part, includes pre-targeted optical imaging and antisense optical imaging methods and products.

Imaging is a powerful diagnostics tool to obtain pathological and physiological information from a subject. This information can subsequently be used for diagnostics and for medical determinations or assessment of treatment regimens. Optical imaging comprises the non-invasive imaging of a subject. As a first step a moiety that can induce or emit a signal is administered to a subject. The moiety can be an agent with total body distribution, like a contrasting reagent, or it can be an agent that specifically binds a certain class of cells or compounds. A subsequent step in optical imaging is the detection of the moiety. The moiety can be detected by the signal emitted by the moiety including, but not limited to: radioactivity, proton resonance, UV, MRI, or fluorescence. In fluorescence optical imaging the moiety is activated by an excitation wavelength and the moiety will emit a fluorescence signal of lesser energy that can be detected. Methods of detection of a signal generated in optical imaging is routine and are known to a person of ordinary skill in the art.

An advantage of optical imaging is that except for the administration of the imaging agent, the technique is non-invasive. In some embodiments of the invention, optical imaging is targeted optical imaging, i.e. the optical imaging agent comprises a moiety or functionality that preferentially binds to a specific target or target entity. In some embodiments of the invention, the presence of a target entity in a cell, tissue, or subject is associated with the presence of a disease or condition in the cell, tissue, or subject. In some embodiments the target entity is associated with cancer.

As used herein, methods of the invention may be carried out in subjects. A subject may be a human or a non-human animal, including, but not limited to a non-human primate, cow, horse, pig, sheep, goat, dog, cat, or rodent. In all embodiments, human subjects are preferred.

The invention relates, in part, to methods for optical imaging of a target using oligomer duplexes. Two aspects of optical imaging described herein are pretargeting optical imaging and antisense optical imaging. Each method utilizes oligomer duplexes for imaging target molecules in a subject. The invention includes, in some aspects, pretargeting optical imaging methods and products and antisense optical imaging methods and products.

### Pretargeting

As used herein, the term “pretargeting” means administration of a binding molecule (that specifically binds a target) to a tissue or subject in advance of administration of a detectably labeled oligomer for labeling the target or target entity. For example, using methods of the invention, a binding protein, such as an antibody or antigen-binding fragment thereof, that specifically binds to a target or target entity may be administered to a subject. The antibody or antigen-binding fragment binds to the target or target entity and a second molecule, one that preferentially binds to the antibody or antigen-binding fragment and comprises a detectable label, is also administered to the subject. The detectably labeled molecule then binds to the bound antibody or antigen-binding fragment, permitting detection of the target or target entity. Using such methods, the detectable label may be more specifically localized to the target or target entity rather than in other non-target regions of the tissue or subject.

Binding molecules may be used in the pre-targeting methods of the invention. A binding molecule as used herein means a molecule that can bind a target of interest. A binding molecule of the invention is attached or conjugated to an oligomer. For pretargeting methods, the binding molecule conjugated to the oligomer is administered to a subject and a duplex that is made up of two hybridized oligomers, one of which has a higher affinity to bind to the oligomer conjugated to the binding molecule, than it has to the oligomer to which it is paired in the duplex. Thus, upon contact with oligomer conjugated to the binding molecule, the duplex will dissociate (e.g., become non-hybridized and single stranded) and the oligomer from the duplex with a higher affinity for the oligomer conjugated to the binding molecule, will hybridize with the oligomer conjugated to the binding molecule, and thus be indirectly bound to the target to which the binding molecule is bound.

As used herein, a binding molecule is a molecule that specifically binds to a target or target entity of the invention. A non-limiting example of a binding molecule that can be used in some embodiments of the invention is an antibody or antigen-binding fragment thereof. Another example of a binding molecule that may be used in methods of the invention is a receptor ligand that can specifically bind to a target molecule on a cell (e.g., a receptor for the ligand). Any suitable molecule that can specifically bind a target or target entity is embraced by the methods of the invention. Such binding molecules may include, but are not limited to, DNA binding proteins, polysaccharide binding proteins, nucleic acids that can bind to a specific nucleic acid sequence, polypeptide binding proteins, synthetic compounds that specifically bind to a target molecule, etc. Those of ordinary skill in the art will recognize that numerous different binding molecules may be used in the methods of the invention.

In certain embodiments, a binding molecule may comprise an antibody or antigen-binding fragment thereof. The antibodies or fragments thereof may be selected for the ability to bind to any antigen or target, including nucleotides, polypeptides, polysaccharides or lipid. In further embodiments, the antibody or antigen-binding fragment thereof is selected from the group consisting of IgG1, IgG2, IgG3, IgG4, IgM, IgA1, IgA2, IgAsec, IgD, IgE or has immunoglobulin constant and/or variable domain of IgG1, IgG2, IgG3, IgG4, IgM, IgA1, IgA2, IgAsec, IgD or IgE. In other embodiments, the antibody is a bispecific or multispecific antibody. In still other embodiments, the antibody is a recombinant antibody, a polyclonal antibody, a monoclonal antibody, a humanized antibody or a chimeric antibody, or a mixture of these. In some embodiments, the antibody is a human antibody, e.g., a monoclonal antibody, polyclonal antibody or a mixture of monoclonal and polyclonal antibodies. Embodiments of antigen-binding fragments are a Fab fragment, a F(ab′)₂ fragment, and a F_{V} fragment CDR3. One of ordinary skill in the art will know how to select an appropriate target antibody or antigen binding fragment thereof for use in the methods of the invention.

In one aspect the invention provides a method for optically imaging a target comprising administering a binding molecule to a subject. In some embodiments the binding molecule specifically binds to the target and comprises a partially hybridized oligomer duplex with a first fluorescent label. In some embodiments the binding molecule comprises a partially hybridized oligomer duplex with a luminescent label. The partially hybridized oligomer duplex comprises an oligomer moiety that is part of the binding molecule and a detectably labeled oligomer hybridized to the oligomer moiety of the binding molecule. The oligomers are partially hybridized because the oligomers differ in length from each other and there is an overhang (unhybridized) part of one of the oligomers.

In some embodiments the partially hybridized oligomer duplex with the first fluorescent label is contacted with an oligomer comprising a second fluorescent label. In some embodiments the second fluorescent label is a luminescent label. In some embodiments the oligomer comprising the second fluorescent label hybridizes to the partially hybridized oligomer duplex. In some embodiments the oligomer comprising the second fluorescent label hybridizes to the unhybridized region of the partially hybridized oligomer duplex. In some embodiments the oligomer comprising the second fluorescent label hybridizes to the duplex region of the partially hybridized oligomer duplex. The invention embraces any hybridization configuration as long as the hybridization results in the second fluorescent label being in close enough proximity to the first fluorescent label to interact with the first fluorescent label. In some embodiments interaction means shifting the fluorescence frequency of the first fluorescent label. In some embodiments interaction means shifting the fluorescence frequency of the second fluorescent label. In some embodiments interaction means quenching the fluorescence of the first or second fluorescent label.

Detection of a shift in fluorescence frequency of the first or second fluorescent label allows for the detection of the binding molecule. Since the binding molecule is bound to a target entity, detection of the binding molecule allows for detection of the target entity and thereby allows for the optical imaging of a target entity in a subject.

### Antisense

In another aspect, the invention provides methods for optical imaging of a target using antisense duplexes. Antisense duplex optical imaging is based on the selective expression or a increased expression of a target mRNA in a specific cell or subset of cells. An antisense duplex comprises an oligomer that is antisense to the target mRNA conjugated to a fluorescent moiety, and a second oligomer comprising a quenching moiety. If an antisense duplex is administered to a subject and the duplex is contacted with the target mRNA, the antisense strand of the antisense duplex will dissociate from the quenching strand and will bind to the target mRNA resulting in an appearance of fluorescence of the now unquenched fluorescent moiety. Thus, fluorescence will be observed in cells that express the target mRNA.

In one aspect, the invention provides a method for optically imaging a target comprising administering a linear antisense-sequence containing oligomer duplex to a subject. In some embodiments the linear oligomer duplex comprises a quenching oligomer and detectably labeled oligomer. The quenching oligomer comprises a quenching moiety, while the detectably labeled oligomer comprises a detectable label. The linear oligomer duplex has a conformation resulting in a quenching of the detectable label by the quenching moiety. In some embodiments the detectable label is a fluorescent label. In some embodiments the detectable label is a luminescent label. In some embodiments quenching comprises a shift in fluorescence frequency.

In some aspects of the invention, upon administration of a linear antisense oligomer duplex, the duplex will dissociate if the duplex is contacted by a specific target nucleic acid. In some embodiments, the specific target nucleic acid will be expressed only in a subset of cells of the subject. In some embodiments the detectably labeled antisense oligomer of the linear oligomer duplex will have a higher affinity for the specific target nucleic acid resulting in binding of the detectably labeled oligomer to the specific target nucleotide and the formation of a duplex between the detectably labeled antisense oligomer and the specific target nucleotide. In some embodiments the antisense quenched oligomer of the linear oligomer duplex will have a higher affinity for the specific target nucleic acid than the antisense oligomer has for the quenching oligomer, which results in binding of the quenched antisense oligomer to the specific target nucleotide and the formation of a duplex between the antisense oligomer and the specific target nucleotide.

The formation of a duplex between the quenched antisense oligomer and the specific nucleic acid target results in an increase in distance between the quenching moiety and the detectable label resulting in unquenching of the detectable label and the appearance of a fluorescent or luminescent signal or a shift in fluorescence frequency and thereby the occurrence of a detectable event. This detectable event will occur only when a specific target nucleic acid is available, thereby providing a method for optical imaging of that target nucleic acid. If the target nucleic acid is expressed in a specific subset of cells, like a cancer cell etc., antisense optical imaging of a target nucleic acid allows for the detection of a target entity, like a cancer cell.

In some aspects of the invention, antisense methods and compounds may be used in optical imaging methods. Antisense as used herein refers to a nucleotide sequence that is complementary to a specific sequence of mRNA. If the specific mRNA is differentially expressed in a particular cell or tissue (versus a control cell or control tissue, respectively), antisense can be used to specifically target that particular cell.

As used herein, the term “antisense oligonucleotide” or “antisense” describes an oligomer or oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. In some embodiments the antisense molecules are designed to hybridize to mRNA expressed only, or in much higher amounts, in target entities, including target cells. Those skilled in the art will recognize that the exact length of the antisense oligonucleotide and its degree of complementarity with its target will depend upon the specific target selected, including the sequence of the target and the particular bases which comprise that sequence.

It is preferred that antisense oligonucleotide be constructed and arranged so as to bind selectively with the target under physiological conditions, i.e., to hybridize substantially more to the target sequence than to any other sequence in the target cell under physiological conditions. Based upon the nucleotide sequences of the target nucleic acid, one of skill in the art can easily choose and synthesize any of a number of appropriate antisense molecules for use in accordance with the present invention. In order to be sufficiently selective and potent for inhibition, such antisense oligonucleotides should comprise at least about 10 and, more preferably, at least about 15 consecutive bases which are complementary to the target, although in certain cases modified oligonucleotides as short as 7 bases in length have been used successfully as antisense oligonucleotides (See Wagner et al., 1995, Nat. Med. 1, 1116-1118). Most preferably, the antisense oligonucleotides comprise a complementary sequence of 20-30 bases. Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted by antisense oligonucleotides. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., 1994, Cell. Mol. Neurobiol. 14, 439-457) and at which proteins are not expected to bind.

Non-limiting examples of antisense oligonucleotides and oligomers with which they may form duplexes for use in methods of the invention are provided in the Examples section. One or ordinary skill in the art will recognize that additional antisense oligomers and oligomers for duplex formation can be designed and used in the methods provided herein.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

### Oligomers

Pretargeting and Antisense methods of the invention utilize oligomers and oligomer duplexes for the identification and labeling of target entities. As used herein, the term “oligomer” or “oligomer moiety” is used to mean one or more nucleotides, i.e. a molecule comprising a sugar (e.g. ribose or deoxyribose) linked to a phosphate group and to an exchangeable organic base, which may be a substituted pyrimidine (e.g. cytosine (C), thymidine (T) or uracil (U)) or a substituted purine (e.g. adenine (A) or guanine (G)). The term “oligomer” also includes “nucleic acid”, “polynucleotides” or “oligonucleotides,” as those terms are ordinarily used in the art. A sequence of nucleotides bonded together, i.e., within a polynucleotide or an oligonucleotide can be referred to as a “nucleotide sequence.” The term “oligomer” also includes nucleosides and polynucleosides (i.e. a nucleotide/polynucleotide without the phosphate). Purines and pyrimidines include, but are not limited to, natural nucleosides. Oligomers of the invention also include “modified oligonucleotides, including, but not limited to, peptide nucleic acid (PNA), locked nucleic acid (LNA), phoshorothioate, and phosphorodiamidate morpholine.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acid molecules has been covalently attached to the oligonucleotide.

Embodiments of synthetic internucleoside linkages are phosphorothioates, alkylphosphonates, phosphorodithioates, phosphate esters, alkylphosphonothioates, phosphoramidates, phosphorodiamidate, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose.

In some embodiments the modified oligonucleotides are phosphorodiamidate morpholino oligomers (Amantana et al., Curr. Opin. Pharmacol. 2005, 5: 550-555).

PNA are synthesized from monomers connected by a peptide bond (Nielsen, P. E. et al. Peptide Nucleic Acids, Protocols and Applications, Norfolk: Horizon Scientific Press, p. 1-19 (1999)). PNAs can be built with standard solid phase peptide synthesis technology. PNA chemistry and synthesis allows for inclusion of amino acids and polypeptide sequences in the PNA design. For example, lysine residues can be used to introduce positive charges in the PNA backbone. All chemical approaches available for the modifications of amino acid side chains are directly applicable to PNA.

PNA has a charge-neutral backbone, and this attribute leads to fast hybridization rates of PNA to DNA. The hybridization rate can be further increased by introducing positive charges in the PNA structure, such as in the PNA backbone or by addition of amino acids with positively charged side chains (e.g., lysines). PNA can form a stable hybrid with DNA molecule. The stability of such a hybrid is essentially independent of the ionic strength of its environment (Orum, H. et al., BioTechniques 19(3):472-480 (1995)), most probably due to the uncharged nature of PNAs. This provides PNA with the versatility of being used in vivo or in vitro. However, the rate of hybridization of PNA that include positive charges is dependent on ionic strength, and thus is lower in the presence of salt.

Several types of PNA designs exist, and these include single strand PNA (ssPNA), bisPNA and pseudocomplementary PNA (pcPNA).

The structure of PNA/DNA complex depends on the particular PNA and its sequence. ssPNA binds to single stranded DNA (ssDNA) preferably in antiparallel orientation (i.e., with the N-terminus of the ssPNA aligned with the 3′ terminus of the ssDNA) and with a Watson-Crick pairing. PNA also can bind to DNA with a Hoogsteen base pairing, and thereby forms triplexes with double-stranded DNA (dsDNA) (Wittung, P. et al., Biochemistry 36:7973 (1997)).

A locked nucleic acid (LNA) is a modified RNA nucleotide. An LNA form hybrids with DNA, which are at least as stable as PNA/DNA hybrids (Braasch, D. A. et al., Chem & Biol. 8(1):1-7 (2001)). Therefore, LNA can be used just as PNA molecules would be. LNA binding efficiency can be increased in some embodiments by adding positive charges to it. LNAs have been reported to have increased binding affinity inherently.

Commercial nucleic acid synthesizers and standard phosphoramidite chemistry are used to make LNAs. Therefore, production of mixed LNA/DNA sequences is as simple as that of mixed PNA/peptide sequences. The stabilization effect of LNA monomers is not an additive effect. The monomer influences conformation of sugar rings of neighboring deoxynucleotides shifting them to more stable configurations (Nielsen, P. E. et al. Peptide Nucleic Acids, Protocols and Applications, Norfolk: Horizon Scientific Press, p. 1-19 (1999)). Also, lesser number of LNA residues in the sequence dramatically improves accuracy of the synthesis. Most of biochemical approaches for nucleic acid conjugations are applicable to LNA/DNA constructs.

Oligomers used in duplexes of the invention may be of different lengths in methods of the invention. The determination of the length of an oligomer of the invention may be based on the differential affinity of an oligomer for another oligomer of different length, versus its affinity for an oligomer closer in length. For example, exemplary oligomer duplexes of the invention may include an oligomer that is 25 nucleotides long hybridized to an oligomer that is 18 nucleotides long. A target oligomer may be 25 nucleotides long, resulting in a higher affinity of the 25 mer from the duplex to hybridize to the target 25 mer rather than for the 25 mer from the duplex to hybridize to the 18 mer of the duplex.

It will be understood that the length of one or more oligomers used in methods of the invention can vary as long as the affinity of an oligomer for its target oligomer is higher than the affinity of the oligomer for the duplex partner or another molecule. Thus, two oligomers in a duplex of the invention for administration to a subject may be 1, 2, 3, 4, 5, 6, 7, 8, 9, 10, 11, 12, 13, 14, 15, 16, 17, 18, 19, 20, or more nucleotides different in length from each other, and a resulting affinity of binding that is such that one of the oligomers from the duplex administered has a higher affinity for a target oligomer than it has for the oligomer it is hybridized to for administration.

In some embodiments the detectably labeled oligomer comprises more nucleotides than the oligomer moiety of the binding molecule. In some embodiments the oligomer moiety of the binding molecule comprises more nucleotides than the detectably labeled oligomer.

The lengths of oligomers used in duplexes and as oligomers attached to targets in methods of the invention can vary and optimal lengths may be determined by one of ordinary skill in the art using routine hybridization methods and parameters. Exemplary oligomers are provided herein include 18 mers and 25 mers. It will be understood that oligomers of lengths from about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more bases in length may be used in the methods of the invention. One of ordinary skill in the art will be able to design and use suitable oligomers for use in duplexes and for attachment to binding molecules using art-known methods.

The level of complementarity between two oligomers may also be adjusted to affect affinity of one oligomer for another oligomer so as to alter duplex formation or dissociation as utilized in the methods of the invention. For example, a oligomer with less complementarity for another oligomer may have a lower binding affinity for that oligomer than to an oligomer that has a higher level of complementarity. Oligomers for use in methods of the invention may differ in complementarity from each other in 1, 2, 3, 4, 5, 6, 7, 8, or more sequence positions.

As used herein, a duplex oligomer is a combination of two oligomers that are hybridized to each other. Oligomers of a duplex of the invention may be of the same length (i.e. have the same number of nucleotides) but are not required to be of the same length. The duplex can be completely hybridized (i.e., have perfect Watson-crick pairing) or can be partially hybridized, or a combination thereof. Partially hybridized encompasses both non-perfect Watson-Crick pairing and overhanging ends. A duplex useful in methods of the invention may include the same type of oligonucleotide backbone (e.g. both DNA) or can have different backbones, e.g., a DNA hybridized to an RNA; a DNA to an LNA, a DNA to an PNA, a PNA to an LNA, etc, including any other combinations. One oligomer of a duplex may include a fluorescent moiety and a second oligomer of the duplex may include a quenching moiety. Prior to administration and prior to contacting the target, oligomers of the invention may be hybridized in such manner to form a duplex that results in quenching of the fluorescent signal.

In some preferred embodiments of the invention, the binding between the oligomers in an oligomer duplex of the invention is linear and does not have loops or hairpins etc. but rather has a linear structure in the regions where the oligomers are bound to each other.

Targeted optical imaging and antisense imaging can be performed by limiting the onset of an optical signal to preferentially occur at the site of its target entity. Limiting signal onset to the site of a target entity can be facilitated by selective induction of a signal in the presence of the target entity. In some embodiments selective induction may occur through use of techniques involving quenching of a fluorescent or luminescent signal. For example, in some methods of the invention, a fluorescent moiety may be delivered to a target entity in a quenched state, and may become unquenched (e.g., become fluorescent and detectable) in the presence of the target entity. Thus, in some embodiments of the invention, detection may be based on the appearance of a fluorescent or luminescent signal that had originally been quenched and the unquenched signal indicates the presence of a target or target entity. In other embodiments of the invention, selective induction may be based on a frequency shift of a fluorescent or luminescent signal [e.g., such as a fluorescent resonance energy transfer (FRET) reaction or a bioluminescent resonance energy transfer (BRET) reaction]. Methods and procedures for utilizing FRET and BRET in detection procedures, including the selection and use of FRET and/or BRET pairs of labels are well known to those of ordinary skill in the art.

### Binding Molecules

Pre-targeting methods of the invention, allow optimization of imaging through the separation of targeting and detection steps. Separation of targeting and detection steps allows for an improved signal to background ratio. As a first step, a binding molecule that can specifically bind to a target entity is administered to a subject. Upon administration to a subject, the binding molecule will preferentially bind to its target, but a portion of the binding molecule may be in non-target locations or regions of the body. The ratio of binding molecule that binds to its target versus the amount of the binding molecule that is located in non-target locations increases as the binding molecule that is not bound to the target is cleared from the subject through normal physiological processes.

A binding molecule specifically binds to a target or target entity. As used herein, the terms “target” and “target entity,” which may be used interchangeably herein, mean the molecule, cell, or other entity that a binding molecule of the invention or an antisense oligomer specifically binds. Specific binding to a target entity means that the binding molecule preferentially binds to the target entity rather binding to other compounds. The binding affinity for a binding molecule or an antisense oligomer for the target entity may be at least 2-fold, at least 5-fold, at least 10-fold, or more than its affinity for another compound. The affinity of an antisense oligomer for its target nucleic acid is higher than the affinity of the antisense oligomer for the oligomer it is hybridized with in a duplex administered to a subject.

A target entity of the invention may be any molecule in a sample or subject that is of interest and may include polypeptides, nucleic acids, polysaccharides, and/or lipid molecules. Targets also include molecules (e.g. drugs) that have been administered to, or otherwise obtained by, a subject. In some embodiments of the invention, a target molecule may be a molecule that is differentially expressed in a cell or tissue of interest versus the expression of the molecule in a cell or tissue that is not a cell or tissue of interest. Thus, a target molecule may be a molecule that is differentially expressed in a cell or tissue of interest versus other cells or tissues, thus permitting one of ordinary skill in the art to use methods of the invention to detect and distinguish such a cell or tissue from other cells or tissues. In some embodiments, a target molecule may be a molecule that is differentially expressed in a cell or tissue that is associated with a disease or condition. In some embodiments, a target molecule may be a molecule that has a specific sequence, or one that has a mutational pattern of expression that is associated with a disease or condition versus a sequence or pattern of expression in a disease-free or condition-free cell or tissue. In some embodiments of the invention, a cell associated with a disease or condition may be a cancer cell. Those of ordinary skill in the art will recognize that numerous target molecules may be used in the methods of the invention and that selection of such a target and selection of a binding molecule that specifically binds to such a target may be made using routine methods known in the art. A molecule that specifically bins to a target of interest may be used as a binding molecule in methods of the invention.

Methods and compositions of the invention can be used to detect the presence of targets in a subject. Those of ordinary skill in the art will recognize the types of targets that may be detected using methods of the invention are not limited to those described herein, but may also include other molecules of interest. Non-limiting examples of polypeptide targets to which methods of the invention may be applied are proteins that are differentially expressed in certain cells or tissues. For example, a protein or nucleic acid that is expressed in a cancer cell or tissue, that is not expressed or is expressed at a different level in a normal (control) cell or tissue may be a target using methods of the invention. Thus, as a non-limiting example, a polypeptide target may be a receptor protein that is overexpressed in a specific tissue or cell. In some embodiments, a cell or tissue associated with a disease or condition may be a cancer cell or tissue.

Non-limiting examples of nucleic acid targets are nucleic acid molecules (e.g., mRNAs, etc.) that are differentially expressed in a cell or tissue of interest versus the expression of the nucleic acid molecule in a cell or tissue that is not a cell or tissue of interest. For example, an mRNA that is known to be differentially expressed in cancer may be a target molecule and may be detected using methods of the invention.

Non-limiting examples of polysaccharides that may be target molecules of the invention are polysaccharides that are differentially expressed on a particular cell type of interest versus the level of the molecule's expression on a cell type that is not the cell type of interest.

A target as used herein is any cell or molecule that has a characteristic that can be distinguished from another cell or molecule. A target can be a cell or molecule that is freely circulating in the body, e.g. in the blood stream, or the target can be part of a specific tissue or located in a specific area of the body. The invention also embraces targets that are not naturally found in a subject, but have been acquired through intervention or exposure (e.g. a drug, or pathogen). A target can be associated with a specific disease or condition, or a target may be a specific subset of molecules or cells present in the body (e.g. T-cells). Exemplary diseases include inflammatory disorders, cancers, autoimmune diseases, neurodegenerative disorders, genetic disorders. Conditions include aging, development and the physiological status of specific targets. Examples of disease include but are not limited to cancer.

As used herein, the term “cancer” refers to an uncontrolled growth of cells that may interfere with the normal functioning of the bodily organs and systems. A cancer cell is a cell that is undergoing, or that has the potential for, uncontrolled cell growth. Cancers that migrate from their original location and seed vital organs can eventually lead to the death of the subject through the functional deterioration of the affected organs. A metastasis is a cancer cell or group of cancer cells, distinct from the primary tumor location resulting from the dissemination of cancer cells from the primary tumor to other parts of the body. At the time of diagnosis of the primary tumor mass, the subject may be monitored for the presence of in transit metastases, e.g., cancer cells in the process of dissemination. Methods of the invention may be used to detect primary and/or metastatic cancer by optical imaging.

As used herein, the term cancer, includes, but is not limited to the following types of cancer, breast cancer, biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chromic myelogenous leukemia, multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Other cancers will be known to one of ordinary skill in the art. In some embodiments of the invention, the cancer is melanoma.

Importantly, levels of a target in a subject can be determined using the pretargeting optical target and/or antisense optical targeting methods of the invention and are advantageously compared to controls according to the invention. A control may be a predetermined value, which can take a variety of forms. It can be a single cut-off value, such as a median or mean. It can be established based upon comparative groups, such as in groups having normal amounts of the target entity and groups having abnormal amounts of the target entity. Another example of comparative groups may be groups having a particular disease (e.g., such as cancer), condition or symptoms and groups without the disease, condition or symptoms. Another comparative group may be a group with a family history of a condition and a group without such a family history. The predetermined value can be arranged, for example, where a tested population is divided equally (or unequally) into groups, such as a low-risk group, a medium-risk group and a high-risk group or into quadrants or quintiles, the lowest quadrant or quintile being individuals with the lowest risk or amounts of the target entity and the highest quadrant or quintile being individuals with the highest risk or amounts of the target entity.

The predetermined value, of course, will depend upon the particular population selected. For example, an apparently healthy population will have a different ‘normal’ range than will a population that is known to have a condition related to an abnormal level of a target entity. Accordingly, the predetermined value selected may take into account the category in which an individual falls. Appropriate ranges and categories can be selected with no more than routine experimentation by those of ordinary skill in the art. As used herein, “abnormal” means not normal as compared to a control. By abnormally high it is meant high relative to a selected control. Typically the control will be based on apparently healthy normal individuals in an appropriate age bracket.

It will also be understood that controls according to the invention may be, in addition to predetermined values, samples of materials tested in parallel with the experimental materials. Examples include samples from control populations or control samples generated through manufacture to be tested in parallel with the experimental samples.

Binding of binding molecules of the invention to a target and/or the binding of an antisense oligomer to its nucleic acid target may be detected using detectable labels that are attached to an oligomer. For example, a detectable label may be attached to a oligomer that is in a duplex when administered to a subject and upon reaching and indirectly binding to the target in the case of a pretargeting oligomer, or directly binding to the target in the case of an antisense oligomer. Binding is indirect in the pretargeting methods because the labeled oligomer binds to the oligomer attached to the binding molecule, not to the target directly. Binding in the antisense optical imaging is directed binding because the detectably labeled antisense oligomer from the administered duplex directly binds (hybridizes) to the target nucleic acid. As used herein the term detectable label, includes, but is not limited to a fluorescent or bioluminescent detectable label. Detection of a fluorescent or bioluminescent detectable label of the invention may be performed using any suitable imaging method, including, but not limited to video microscopy, real-time imaging, or other means that permit imaging of detectable labels of the invention.

In some aspects of the invention, a detectable label may be administered in a form that is not detectable, (e.g., is quenched or not fluorescent or luminescent at an appropriate detection wavelength) until oligomer that includes the label moiety is in close proximity to a target of the invention. For example, a fluorescent molecule may be administered in conjunction with a light-quenching molecule such as BHQ3 or Iowa black, thus quenching fluorescence emitted from the fluorescent molecule as long as it is in proximity to the quencher. An example of such a quenched oligomer of the invention may be an oligomer that is in a duplex with a second oligomer, the first of which has a detectable label and the second of which has a quenching moiety such that the detectable label of the first oligomer is quenched when the two oligomers are hybridized to each other in the duplex. Unquenching of such a detectable label may occur when the duplex dissociates and the detectably labeled oligomer hybridizes to an oligomer bound to the binding molecule bound to the target. Thus, the label will be detectable when the labeled oligomer indirectly binds to the target. Similarly, a detectably labeled antisense oligomer that is quenched when bound in a duplex for administration, will be unquenched when it is no longer hybridized to the quenching oligomer and bound to its target.

In some embodiments, a fluorescent or luminescent molecule may be administered in conjunction with another fluorescent molecule such that FRET and/or BRET methods result in a wavelength of light emission that shifts when the first fluorescent molecule is no longer in close enough proximity to the second fluorescent molecule. In each case, a change in the level or wavelength of detectable light emitted from the detectably labeled oligomer of the invention upon binding to a target can be used to detect the presence of a target in a sample or subject. In each case, binding of a detectably labeled molecule to the target results in a change in light emission that can be detected as a measure of the presence and/or amount of a target in a sample or subject.

Any suitable fluorescent moiety can be used as a detectable label in the methods of the invention. Non-limiting examples of fluorescent molecules are POPO-1, TOTO-3, TAMRA, Alexa 546, Alexa 647, fluorescein, rhodamine, tetramethylrhodamine, R-phycoerythrin, Cy-3, Cy-5, Cy-7, Texas Red, Phar-Red, allophycocyanin (APC), fluorescein amine, eosin, dansyl, umbelliferone, 5-carboxyfluorescein (FAM), 2′7′-dimethoxy-4′5′-dichloro-6-carboxyfluorescein (JOE), 6 carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), 5-(2′-aminoethyl) aminonaphthalene-1-sulfonic acid (EDANS),4-acetamido-4′-isothiocyanatostilbene-2,2′ disulfonic acid, acridine, acridine isothiocyanate, r-amino-N-(3-vinylsulfonyl)phenylnaphthalimide-3,5, disulfonate (Lucifer Yellow VS), N-(4-anilino-1-naphthyl)maleimide, anthranilamide, Brilliant Yellow, coumarin, 7-amino-4-methylcoumarin, 7-amino-4-trifluoromethylcouluarin (Coumarin 151), cyanosine, 4′,6-diaminidino-2-phenylindole (DAPI), 5′,5″-diaminidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid, 4-dimethylaminophenylazophenyl-4′-isothiocyanate (DABITC), eosin isothiocyanate, erythrosin B, erythrosin isothiocyanate, ethidium, 5-(4,6-dichlorotriazin-2-yl) aminofluorescein (DTAF), QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron® Brilliant Red 3B-A), lissamine rhodamine B sulfonyl chloride, rhodamine B, rhodamine 123, rhodamine X, sulforhodamine B, sulforhodamine 101, sulfonyl chloride derivative of sulforhodamine 101, tetramethyl rhodamine, riboflavin, rosolic acid, and terbium chelate derivatives.

In some embodiments of the invention pre-targeting may be facilitated by a binding molecule that comprises an oligomer moiety, and detection of the target may be facilitated by a detectably labeled oligomer that can preferentially hybridize to the oligomer moiety of the binding molecule rather than to the oligomer with which it was duplexed at the time of administration to the subject. In some embodiment, detection of such a detectably labeled oligomer may result from removal of a quenching oligomer resulting in a shift in the frequency of the detectable label when the duplex comprising the detectably labeled oligomer dissociates.

In some embodiments of the invention, detection of a target may be based on the to appearance of a fluorescent or luminescent signal that had originally been quenched, e.g., the unquenching of a quenched signal. In some embodiments of the invention, a signal may be originally quenched because the detectable label is in close proximity to a quenching moiety. As used herein, a quenching moiety is a quenching molecule that is attached to a molecule of the invention.

In some embodiments of the invention, a quenching moiety is an absorbance moiety that does not fluoresce and is able to quench the fluorescent signal of the fluorescent moiety or detectable label. A dark quencher absorbs the fluorescent energy from the fluorophore, but does not itself fluoresce. Rather, the dark quencher dissipates the absorbed energy, typically as heat. Non-limiting examples of dark or non-fluorescent quenchers are Dabcyl, Black Hole Quenchers, Iowa Black, BH3Q, QSY-7, AbsoluteQuencher, Eclipse non-fluorescent quencher, and metal clusters such as gold nanoparticles. Those of ordinary skill in the art will be able to identify and use additional dark quenchers in the methods of the invention without undue experimentation.

Detection of a target using methods of the invention may be based on the unquenching of a fluorescent or luminescent signal when the detectable label is in close enough proximity to the target. As used herein, the term “signal” means the light (e.g., fluorescence or luminescence) emitted by a detectable label. In some embodiments quenching may be facilitated by introduction of a quenching oligomer that comprises a quenching moiety that quenches the detectable label when in close enough proximity to the detectable label. A quenching oligomer may be hybridized to a detectably labeled oligomer in such a way that the quenching moiety of the quenching oligomer is in close enough proximity to the detectable label of the detectably labeled oligomer to quench the signal of the detectable label. Examples of distances between a quenching moiety and a detectable label on an oligomer of the invention are provided herein in the Examples section and those of ordinary skill in the art will recognize routine methods to determine and to optimize the distance between a detectable label and a quencher for use in methods of the invention. The use of quenching and fluorescence pairs is well known in the art and those of ordinary skill in the art will be able to utilize and optimize the use of such pairs in methods of the invention without undue experimentation.

In some embodiments, the signal of the detectable label of a quenched first linear oligomer duplex is quenched by at least 1%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, including all percentage in between each percentage listed. In some embodiments a detectable label is on the 5′ end of a detectably labeled oligomer. In some embodiments a detectable label is on the 3′ end of a detectably labeled oligomer. In some embodiments a quenching moiety is on the 3′ end of a quenching oligomer and in certain embodiments a quenching moiety is on the 5′ end of a quenching oligomer.

### FRET/BRET

In some embodiments of the invention, detection of a target may be based on a shift in fluorescence frequency of a fluorescent or luminescent moiety of the detectably labeled oligomer. Examples of detection methods that utilize such a shift are FRET and BRET method, both of which are methods routinely used in the art. Thus, in some embodiments, a detectable label is a fluorescence donor or donor fluorophore and the quencher is an fluorescence acceptor or acceptor fluorophores. In some embodiments the donor and acceptor fluorophores form a FRET (fluorescence resonance energy transfer) pair. If the donor fluorophore is excited, for instance by a laser light, a portion of the energy absorbed by the donor is transferred to acceptor fluorophore, if the acceptor fluorophores are spatially close enough to the donor molecules (i.e., the distance between them must approximate or be less than the Forster radius or the energy transfer radius). Once the acceptor fluorophore absorbs the energy, it in turn fluoresces in its characteristic emission wavelength, resulting in a shift in frequency of fluorescence. Examples of FRET donors include Alexa 488, Alexa 546, BODIPY 493, Oyster 556, Fluor (FAM), Cy3 and TMR (TAMRA). Examples of FRET acceptors include Cy5, Alexa 594, Alexa 647 and Oyster 656.

FRET generally requires only one excitation source (and thus wavelength) and only one detector. The detector may be set to either the emission spectrum of the donor or acceptor fluorophore. The detector is set to the donor fluorophore emission spectrum if FRET is detected by quenching of donor fluorescence. Alternatively, the detector is set to the acceptor fluorophore emission spectrum if FRET is detected by acceptor fluorophore emission. In some embodiments, FRET emissions of both donor and acceptor fluorophores can be detected. In still other embodiments, the donor is excited with polarized light and polarization of both emission spectra is detected.

In other embodiments, the resonance energy transfer signal is due to luminescence to resonance energy transfer (LRET; Mathis, G. Clin. Chem. 41, 1391-1397, 1995) and the donor moiety is a luminescent moiety. In some embodiments the luminescent moiety is a chemiluminescent moiety (CRET; Campbell, A. K., and Patel, A. Biochem. J. 216, 185-194, 1983). In some embodiments the luminescent moiety is bioluminescent moiety (BRET; Xu, Y., Piston D. W., Johnson, Proc. Natl. Acad. Sci., 96, 151-156, 1999).

In some embodiments where the resonance energy signal is due to chemiluminescence, the donor moiety can be a lanthanide like Europium or Terbium. Furthermore, in some embodiments where the resonance energy signal is due to chemiluminescence, the donor moiety can be a lanthanide chelate such as DTPA-cytosine, DTPA-cs124, BCPDA, BHHCT, Isocyanato-EDTA, Quantum Dye, or W1024 and the acceptor moiety can be Cy-3, ROX or Texas Red. In some embodiments, due to the range of effective resonance energy transfer of the lanthanide chelate, multiple acceptor moieties may be employed. The donor moiety can be a lanthanide chelate and the acceptor moiety can be a phycobiliprotein. In certain embodiments, the phycobiliprotein is Red Phycoerythrin (RPE), Blue Phycoerythrin (BPE), or Allophycocyanin (APC).

In BRET the donor protein is a bio-luminescent protein and the acceptor protein is a fluorescent protein. In some embodiments the donor luminescent protein is Renilla luciferase or firefly luciferase. In some embodiments the fluorescent acceptor protein is a green, red, cyan or yellow fluorescent protein.

In the methods of the invention, a binding molecule or a target is contacted with an oligomer. As used herein, the term “contacting a molecule” with an oligomer, may mean contacting a binding molecule with an oligomer (e.g., with a quenched first linear oligomer duplex) and may also mean contacting a target molecule with an antisense oligomer from a duplex both of which include bringing the two entities into close enough proximity to allow them to interact with each other. As used herein the term “interact” means binding or hybridization of one or more oligomers of the quenched first linear oligomer duplex with the oligomer moiety of the binding molecule or binding or hybridization of the antisense oligomer to the target molecule. Once a quenched first linear oligomer complex is contacted with a binding molecule, the detectably labeled oligomer of the first linear oligomer duplex hybridizes to the oligomer moiety of the binding agent, forming a linear duplex. The detectably labeled oligomer will hybridize to the oligomer moiety of the binding molecule because the detectably labeled oligomer has a higher affinity to form a linear duplex with the oligomer moiety of the binding molecule than the detectabley labeled oligomer has to form a linear duplex with the quenching oligomer. Binding of the detectably labeled oligomer to the oligomer moiety of the binding molecule allows for the detection of unquenched detectable label in the subject. Because the binding molecule is bound to a target entity, detection of the detectable label allows for detection of the target entity and thereby allows for the optical imaging of a target entity in a subject. Similarly, a detectably labeled antisense oligomer of an oligomer duplex may preferentially hybridize with a target nucleic acid sequence, thus forming a duplex with the target nucleic acid sequence and detectably labeling the target sequence.

The detectably labeled oligomer of a first linear oligomer duplex of the invention may bind to the oligomer moiety of the binding molecule because it has a higher affinity for the oligomer moiety of the binding agent than the affinity of the first linear oligomer duplex for the quenching oligomer of the quenched first linear oligomer duplex. Similarly, a detectably labeled antisense oligomer of an antisense duplex may bind to the target nucleic acid because it has a higher affinity for the target nucleic acid than for the other oligomer (the quenching oligomer) of the duplex. As used herein, for a first oligomer to have a “higher affinity” for a second oligomer than the first oligomer has to a third or other oligomer, means that the first oligomer will preferably hybridize to the second oligomer rather than to the third or other oligomer. In some embodiments higher affinity for the oligomer moiety of the binding agent than for the quenching oligomer of the quenched first linear oligomer duplex means that the linear oligomer duplex of the detectably labeled oligomer and the oligomer moiety of the binding molecule has a higher melting temperature than the linear oligomer duplex of the detectably labeled oligomer and the quenching oligomer. Similarly, in some embodiments higher affinity for the antisense oligomer moiety for the target nucleic acid (e.g., the target oligomer) than for the quenching oligomer of the antisense duplex means that the linear oligomer duplex of the detectably labeled oligomer and its antisense target oligomer has a higher melting temperature than the linear antisense oligomer duplex of the detectably labeled antisense oligomer and the quenching oligomer. In some embodiments a higher melting temperature means that the oligomer duplex has more hydrogen bonds between the hybridizing oligomers. In some embodiments the duplex is comprised of two single stranded oligomers. In forming and maintaining a duplex, oligomers hybridize through Watson-Crick binding. In some embodiments a higher melting temperature comprises more complementing Watson-Crick base pair interactions. However, the invention embraces all modes of oligomer hybridization including binding of single-strand oligomers to duplexes. In some embodiments the binding between the oligomers includes Hoogsteen binding.

Components used in optical imaging and diagnostics, including for example, binding molecules, quenched first linear oligomer duplexes, and linear oligomer duplexes can be administered to a subject by any suitable mode. As used herein, binding molecules, quenched first linear oligomer duplexes, quenched first linear oligomer duplexes, linear oligomer duplexes and/or other compounds administered to a subject in a method of the invention may be referred to herein as pretargeting or antisense targeting compounds of the invention.

A pretargeting or antisense targeting compound of the invention may be administered in an effective amount to permit optical imaging of a target of interest in a subject. Typically an effective amount of a compound that permits detection of the target in a manner that is diagnostically useful and sufficient for the purposes for which the methods of the invention are utilized. Generally, an effective amount of a duplex or binding agent/oligomer compound, etc., will be determined in practice and/or using clinical trials, e.g., establishing an effective dose for a test population in a study. In some embodiments, an effective amount will be an amount that results in a desired response, e.g., visualization and/or detection of a target. Thus, an effective amount may be the amount that when administered permits imaging of a target.

An amount that is an effective amount will vary with the particular type of target to be detected, binding molecule used, the age and physical condition of the subject being tested, the presence of known disease and/or disorders in the subject (e.g., cancer, cardiac disease, coronary artery disease, etc.), the nature of any concurrent therapy, the specific route of administration, and additional factors within the knowledge and expertise of the health practitioner. For example, an effective amount may depend upon the degree of cancer in the individual and/or the location of the cancer in the individual subject to be tested. Such factors are well known to those of ordinary skill in the art and can be addressed with no more than routine experimentation.

It is generally preferred that a maximum dose of a pretargeting or antisense targeting compound of the invention be used, that is, the highest safe dose according to sound medical judgment. It will be understood by those of ordinary skill in the art, however, that a patient may insist upon a lower dose or tolerable dose for medical reasons, psychological reasons or for virtually any other reasons. The dosage of one or more pretargeting or antisense targeting compounds of the invention administered to a subject can be chosen in accordance with different parameters, in particular in accordance with the mode of administration used and the state of the subject. In the event that a response in a subject is insufficient at the initial doses applied, higher doses may be employed to the extent that patient tolerance permits.

A pretargeting and/or antisense compound used in the foregoing methods preferably are sterile and contain an effective amount of a pretargeting and/or antisense targeting compound that will permit sufficient imaging of a target in a subject.

A pretargeting and/or antisense compound of the invention may be administered alone, in combination with each other, and/or in combination with other imaging agents or regimens that are administered to subjects.

A pretargeting and/or antisense targeting compound dosage may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations. The absolute amount will depend upon a variety of factors, including the material selected for administration, whether the administration is in single or multiple doses, and individual subject parameters including age, physical condition, size, weight, and the stage of the disease or condition. These factors are well known to those of ordinary skill in the art and can be addressed with no more than routine experimentation.

A pretargeting and/or antisense targeting compound may be administered as a pharmaceutical composition. A pharmaceutical composition of the invention, for use in the foregoing methods preferably are sterile and contain an effective amount of a pretargeting or antisense targeting compound that will permit suitable imaging, e.g., a level that produces the desired response in a unit of weight or volume suitable for administration to a patient.

The doses of a pretargeting and/or antisense targeting compound, or other pharmaceutical compound of the invention administered to a subject can be chosen in accordance with different parameters, in particular in accordance with the mode of administration used and the state of the subject. Other factors include the desired period of imaging. In the event that an imaging response in a subject is insufficient at the initial doses applied, higher doses (or effectively higher doses by a different, more localized delivery route) may be employed to the extent that patient tolerance permits.

Various modes of administration will be known to one of ordinary skill in the art that effectively deliver a pretargeting and/or antisense targeting compound to a desired tissue, cell or bodily fluid. Methods for administering a pretargeting and/or antisense targeting compound of the invention may be topical, intravenous, oral, intracavity, intrathecal, intrasynovial, buccal, sublingual, intranasal, transdermal, intravitreal, subcutaneous, intramuscular and intradermal administration. The invention is not limited by the particular modes of administration disclosed herein. Standard references in the art (e.g., Remington's Pharmaceutical Sciences, 18th edition, 1990) provide modes of administration and formulations for delivery of various pharmaceutical preparations and formulations in pharmaceutical carriers. Other protocols which are useful for the administration of a pretargeting and/or antisense targeting compound of the invention will be known to one of ordinary skill in the art, in which the dose amount, schedule of administration, sites of administration, mode of administration (e.g., intra-organ) and the like vary from those presented herein.

Administration of a pretargeting and/or antisense targeting compound of the invention to mammals other than humans, e.g., for testing purposes or veterinary therapeutic purposes, is carried out under substantially the same conditions as described above.

When administered, the pharmaceutical preparations of the invention may be applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally, therapeutic agents.

A pretargeting and/or antisense targeting compound of the invention may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the pretargeting and/or antisense targeting compound of the invention, and with each other, in a manner such that there is no interaction which would substantially impair the desired imaging efficacy.

A pharmaceutical composition of the invention may contain suitable buffering agents, as described above, including: acetate, phosphate, citrate, glycine, borate, carbonate, bicarbonate, hydroxide (and other bases) and pharmaceutically acceptable salts of the foregoing compounds.

A pharmaceutical composition of the invention, also may contain, optionally, suitable preservatives, such as: benzalkonium chloride; chlorobutanol; parabens and thimerosal. The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration may comprise a pretargeting and/or antisense targeting compound of the invention. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

A pretargeting and/or antisense targeting compound of the invention, may be administered as one dose, or multiple doses.

Any duplex of the invention can be administered before or simultaneously with the binding molecule or in preferred embodiments, the duplex can be administered after the binding molecule has been administered. In some embodiments the duplex is administered after the binding molecule is bound to the target and the surplus of unbound binding molecule has been removed from the subject by natural physiological processes. The duplex may be administered up to 1, 2, 5, 10, 15, 20, 30, 60, 120 minutes or up to 4, 6, 12, 24, 48 hours or any time after the binding molecule has been administered. In addition, multiple doses of duplex may be administered. The duplex and the binding molecule may be administered through the same routes, e.g., both intravenously, or they may be administered through different methods, e.g. the binding molecule may be administered intravenously, while the duplex is administered orally.

Methods and/or kits of the invention can be used to obtain useful prognostic information by providing an early indicator of disease onset, progression, and/or regression. The invention includes methods to monitor the onset, progression, or regression of disease in a subject by, for example, optically imaging the target at specific time points. A subject may be suspected of having the disease or may be believed not to have disease and in the latter case, the optical image acquired at the initial time point may serve as a normal baseline level for comparison with subsequent imaging events.

Onset of a condition is the initiation of the changes associated with the condition in a subject. Such changes may be evidenced by physiological symptoms, or may be clinically asymptomatic. For example, the onset of the disease may be followed by a period during which there may be disease-associated pathogenic changes in the subject, even though clinical symptoms may not be evident at that time. The progression of a condition follows onset and is the advancement of the pathogenic (e.g. physiological) elements of the condition, which may or may not be marked by an increase in clinical symptoms. In contrast, the regression of a condition may include a decrease in physiological characteristics of the condition, perhaps with a parallel reduction in symptoms, and may result from a treatment or may be a natural reversal in the condition.

Methods and compositions of the invention are also useful to characterize levels of a target entity in a subject by monitoring changes in the amount of the target entity over time. For example, it is expected that an increase in a cancer-associated target entity may correlate with an increase in the progression of cancer. Accordingly one can monitor the target entity's levels over time to determine if its levels in the subject are changing. Changes in the level of a target entity of greater than 0.1% may indicate an abnormality or change in disease or condition status of a subject. Preferably, the change in a level of a target entity, which indicates an abnormality, is greater than 0.2%, greater than 0.5%, greater than 1.0%, 2.0%, 3.0%, 4.0%, 5.0%, 7.0%, 10%, 15%, 20%, 25%, 30%, 40%, 50%, or more. Reductions in amounts of a target entity over time may indicate remission of a disease or condition if the target is one for which increased expression is characteristic for an increased severity of the disease or condition. Similarly, a reduction in an amount of a target entity over time may indicate onset or progression of a disease or condition if the disease or condition is one characterized by a decreased expression of the target entity.

The methods and products of the invention may also be used in diagnostic methods to determine the effectiveness of treatments for diseases or disorders characterized by alterations in expression of the target entity. The “evaluation of treatment” as used herein, means the comparison of a subject's levels of a target entity measured at different times, preferably at least one day apart. In some embodiments, the time to obtain the second measurement from the subject is at least 1, 2, 3, 4, 5, 6, 7, 8, 9, 10, 11, 12, 13, 14, 15, 16, 17, 18, 19, 20, 21, 22, 23, 24, 36, 48, 72, 96, 120, 250, 350, 450, 550, or more (including all intervening integers) days after obtaining the first sample from the subject.

The methods and compositions of the invention may be used to allow the comparison of levels of a target entity at different times, which allows evaluation of the status of the subject's condition and/or allows evaluation of the efficacy of treatments of the disease or condition in the subject. The comparison of a subject's levels of a target entity measured in the subject at different times provides a measure of changes in the status of the disease or condition, and permits assessment of the effectiveness of a treatment, age, or other change in status of the subject.

As used herein, the term “diagnose” means the initial recognition of cancer or a precancerous condition in a cell, tissue, and/or subject and also may mean determination of the status or stage of cancer or a precancerous condition in the cell, tissue, and/or subject. For example, a diagnosis of cancer or a precancerous condition in a subject using a methods of the invention may include the determination of the stage of cancer, and/or pathogenic features of cancer in the subject. The diagnosis may be based on the detection cancer cells or to other targets by optical imaging.

Diagnosis using optical imaging methods of the invention may be combined with diagnosis methods routine in the art. Such diagnostic assays include but are not limited to histopathology, immunohistochemistry, flow cytometry, cytology, patho-physiological assays, including MRI and tomography, and biochemical assays. Biochemical assays include but are not limited to mutation analysis, chromosomal analysis, ELISA analysis of specific proteins, platelet count etc. Those of ordinary skill in the art will be aware of numerous diagnostic and staging protocols and parameters that are routinely utilized in the art.

Also within the scope of the invention are kits comprising the components of the invention and instructions for use. Kits of the invention may be useful for diagnosing a disease or condition. Kits of the invention may include one or more components for optically imaging a target entity. One embodiment of such a kit may include a binding molecule and a quenched first linear oligomer duplex. Another embodiment of such a kit may include a binding molecule and an oligomer comprising a fluorescent label. Another embodiment of such a kit may include a linear oligomer duplex. In some embodiments, a kit of the invention may include components for the administration of the binding molecules, oligomers and other components, including buffers and pharmaceutical compositions

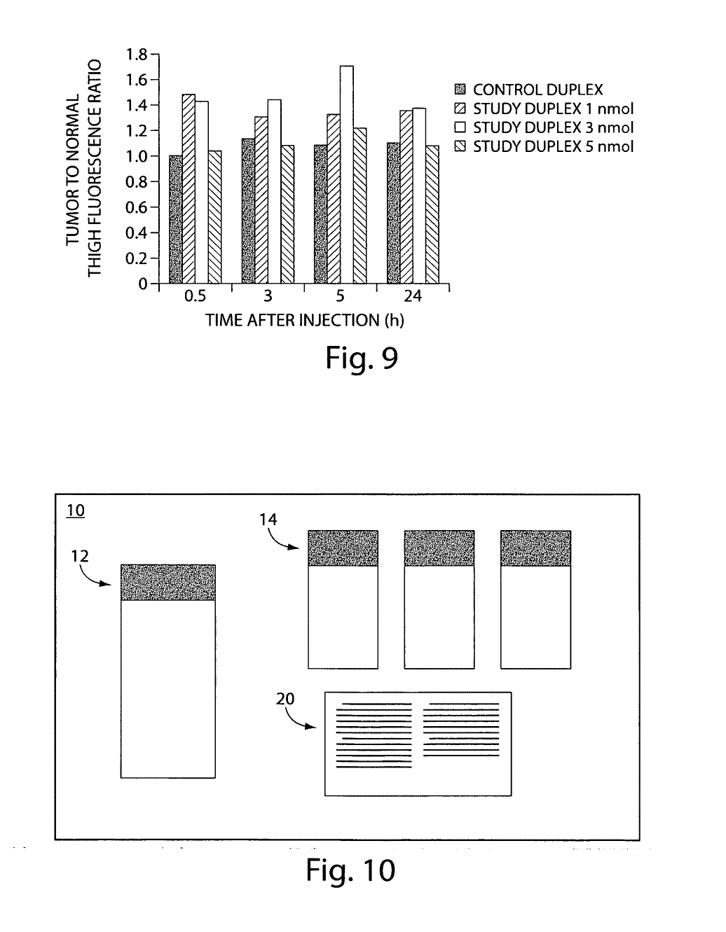

One embodiment for a kit for diagnosing cancer (10=kit, 12=components for optical imaging; 14=additional components; 20=instructions) is depicted in FIG. 10.

A kit may comprise a carrier being compartmentalized to receive in close confinement therein one or more container means or series of container means such as test tubes, vials, flasks, bottles, syringes, or the like. A first of said container means or series of container means may contain a binding molecule. A second container means or series of container means may contain a linear oligomer duplex.

A kit of the invention may also include instructions. Instructions typically will be in written form and will provide guidance for carrying-out the methods embodied by the kit and for making a determination based upon that assay.

The present invention is further illustrated by the following Examples, which in no way should be construed as further limiting. The entire contents of all of the references (including literature references, issued patents, published patent applications, and co-pending patent applications) cited throughout this application are hereby expressly incorporated herein by reference in their entirety.

### EXAMPLES

### Example 1

### Introduction

Pretargeting with radioactivity has significantly improved tumor to normal tissue radioactivity ratios over conventional antibody imaging in both animal studies and clinical trails. This laboratory has investigated DNA analogues such as phosphorodiamidatemorpholinos (MORFs) for pretargeting using technetium-99m (99 mTc) for detection. However, the unique properties of florescence activation and quenching combined with oligomers with their unique properties of hybridization may be particularly useful when used together for pretargeting with optical detection. The use of linear fluorophore-conjugated oligomer duplexes have been little used in animals, and to our knowledge, have not previously been considered for pretargeting applications.

For these studies a MORF/cDNA pair was selected such that when hybridized, the fluorescence of the Cy5.5-conjugated 25 mer MORF (Cy5.5-MORF25) is inhibited with a BHQ3-conjugated 18 mer complementary DNA (BHQ3-cDNA18). The short BHQ3-cDNA18 was selected to dissociate in the presence of a long cMORF25 in the pretargeted tumor, thus, releasing the inhibitor from the Cy5.5 emitter. In this manner, the Cy5.5 fluorescence was inhibited everywhere but in the target. The dissociation was first examined in vitro by adding the duplex to the cMORF25 both in solution and immobilized on polystyrene microspheres and by surface plasmon resonance (SPR). Thereafter, biotinylated cMORF25 immobilized on streptavidin polystyrene microspheres were administered intramuscularly in one thigh of hairless SKH-1 mice as target while an identical weight of the identical microspheres but without the cMORF25 was administered in the contralateral thigh as control. The animals then received IV the Cy5.5-MORF25/BHQ3-cDNA18 duplex or equal molar dosage of single-chain Cy5.5-MORF25 and were imaged. The SPR studies showed that the immobilized cDNA18 rapidly captured the flowing MORF25 to provide a duplex with a slow dissociation rate constant. Furthermore, when cMORF25 was next allowed to flow over the now immobilized duplex, the cDNA18 was unable to prevent dissociation of the heteroduplex and the formation and release of the cMORF25-MORF25 homoduplex. Images of animals obtained soon after receiving the Cy5.5-MORF25 singlet showed intense whole body fluorescence obscuring the target thigh. However, only 5 minutes after receiving the Cy5.5-MORF25/BHQ3-cDNA18 duplex, the target thigh was clearly visible along with only the kidneys.

This study of optical pretargeting provides a proof of concept that oligomer pretargeting found to be useful with radioactivity detection is applicable with fluorescent detection as well. In addition, the results demonstrate that by using linear oligomers for optical pretargeting, chain lengths (and base sequences) may be manipulated to provide duplexes with stabilities and fluorescence inhibition optimized for pretargeting and other in vivo applications of optical imaging.

### Background

Next to radioactivity methods, optical imaging may be the most sensitive of noninvasive in vivo imaging modalities, at least, as concerns surface tissues (Ke, S. et al., Cancer Res 63:7870-7875, 2003; Ntziachristos, V. et al., Eur Radiol 13:195-208, 2003). Optical (fluorescent and bioluminescent) imaging is an extremely useful research tool with small animals and is increasingly being considered as a clinical modality. One major advantage of optical imaging methods over radioactivity methods is the possibility of turning the signal on and off through the judicious use of fluorescence resonance energy transfer (FRET) (Emptage, N. J, Curr Opin Pharmacol 1:521-525 2001; McIntyre, J. O. et al., Biochem J 377:617-628, 2004). The use of FRET to inhibit and enhance fluorescence in DNA-based molecular beacons is common but primarily in vitro and using hairpin DNAs (Molenaar, C. et al., Nucleic Acids Res 29:E89-E89 2001; Tyagi, S. et al., Biophys J 87:4153-4162, 2004).

To our knowledge, the use of linear fluorophoreconjugated oligomer duplexes have not previously been considered for pretargeting applications. For reasons of simplicity and cost, this investigation was designed for shorter and linear oligomer heteroduplexes instead of hairpins. The Cy5.5 emitter was selected because its emission in the near infrared is suitable for in vivo use (Cheng, Z. et al., Bioconjug Chem 16:1433-1441, 2005). The heteroduplex consisted of a 25-mer phosphorodiamidate morpholino (MORF) oligomer covalently conjugated with the Cy5.5 emitter on its 3′ equivalent end (i.e., Cy5.5-MORF25) hybridized to an 18-mer complementary phosphorothioate DNA with the black hole inhibitor BHQ3 on its 5′ end (i.e., BHQ3-cDNA18). By positioning the emitter and inhibitor in close proximity, the duplex will remain “dark”. However, the Cy5.5 will fluoresce when the heteroduplex dissociates in proximity to its target 25 mer complementary MORF (i.e., cMORF25) to form the Cy5.5-MORF25/cMORF25 homoduplex. Accordingly, the fluorescence should, in principle, be restricted only to the target. These concepts were tested herein in a mouse microsphere model after successfully demonstrating the dissociation of duplex in the presence of its target in vitro and by surface plasmon resonance (SPR).

### Methods

All MORFs and cMORFs [collectively: (c)MORFs] were purchased prepurified with amine modification on the 3′ equivalent end (Gene-Tools, Corvallis, Oreg.) and were used as received. The MORF25 used in this investigation was identical to the MORF in continuous use in this laboratory (5′ equivalent-TGGTGGTGG GTGTACGTCACAACTA (SEQ ID NO:1)-C(O)—CH₂—CH₂—NH₂) (He, J. et al., J Nucl Med 45:1087-1095, 2004). The phosphorothioate BHQ3-cDNA18 (5′-BHQ3-linker-TAGTTGTGACGTACACCC (SEQ ID NO:2)) with the glycolate linkage was purchased from Biosearch Technologies, Inc. (Novato, Calif.). The biotinylated DNA18 and cDNA18 for SPR studies were purchased from Operon Biotechnologies, Inc. (Huntsville, Ala.). Cy5.5 monofunctional N-hydroxysuccinimide (NHS) ester (Cy5.5-NHS) was purchased from Amersham Biosciences (Piscataway, N.J.) and conjugated with MORF25 according to the manufacturer's recommended procedure. The 1-μm colorless streptavidin coated carboxylated polystyrene microspheres, with a free biotin binding capacity of 3.5 μg biotin/mg microspheres, was purchased from Polysciences, Inc. (Warrington, Pa.). All other chemicals were reagent grade and were used without purification.

### Surface Plasmon Resonance (SPR)

SPR was performed on a BIAcore 2000 (BIAcore, Piscataway, N.J.) instrument operating at room temperature as previously described (He, J. et al., Bioconjug Chem 16:1098-1104, 2005). As before, the biotinylated cDNA18 at 20 nM was added in 5-10 μl aliquots to a new streptavidin dextran coated sensor chip (SA) at a flow rate of 20 μl/min only until a response of about 100 (±10) RUs was reached. Hybridization of an immobilized oligomer with its complementary oligomer in the running buffer will be apparent by an increase in response. The absence of mass transfer effects was confirmed by running separately one concentration of free MORF at three different flow rates (10, 30, and 75 μL/min) and demonstrating identical response and curve shape for all three sensorgrams. Solutions of free cMORF25 and MORF25 were prepared at 40 nM in the same running buffer (10 mM HEPES, 150 mM NaCl, 3.4 mM Na2EDTA, 0.005% P20, pH 7.4). The duplex MORF25/cDNA18 was first formed on the chip surface by injecting the MORF25 solution to the active (cDNA18) or control (DNA18) surfaces at a flow rate of 30 μl/min. Thereafter, the cMORF25 solution was injected to monitor the dissociation kinetics. The chip surface was regenerated with 100 mM HCl. To correct for nonspecific binding and refractive index changes, the response from the control surface was subtracted from that obtained from the active surface. A minor baseline drift resulting from a slow dissociation of the complex on the active and control surfaces was eliminated by also subtracting sensorgrams obtained following the injection of running buffer alone (He, J., et al., Bioconjug Chem 14:1018-1023, 2003).

### In Vitro Studies

The ability of the BHQ3-cDNA18 quencher to inhibit the fluorescence of Cy5.5-MORF25 was evaluated in solution using a SpectraMax M5/M5e Microplate Reader (Molecular Devices Corporation, Sunnyvale, Calif.). Solutions of Cy5.5-MORF25 and BHQ3-cDNA18 were prepared in Dulbecco's phosphate-buffered saline (PBS), pH 7, at 20-100 μM. Thereafter, BHQ3-cDNA18 was added to 0.1 nmol of Cy5.5-MORF25 at two- and five-fold molar excess and the fluorescence intensity measured as below.

To study the release of Cy5.5-MORF25 from the Cy5.5-MORF25/BHQ3-cDNA18 duplex in the presence of cMORF25, cMORF25 was first immobilized on microspheres by adding about 15 nmol of biotinylated cMORF25 to 300 μl of the streptavidincoated microspheres followed by three washings with PBS. Thereafter, 0.1 nmol of the duplex was added to 100 μl of the above cMORF25 microspheres or to the same concentration of microspheres without the cMORF25 as control. After incubation for another 30 minutes, the microspheres were again washed three times with PBS and fluorescence intensity measured in a 96-well plate with excitation at 675 nm and detection at 694 nm at the following instrument set-up: top reading, 20-second mixing time, wavelength cut-off at 695 to 700 nm. As additional controls, the duplex without the quencher (i.e., Cy5.5-MORF25/cDNA18 and Cy5.5-MORF25 alone were also added to the microspheres.

### In Vivo Imaging

In vivo fluorescence imaging was performed on an IVIS 100 small animal imaging system (Xenogen, Alameda, Calif.) using a Cy5.5 filter set. Identical illumination settings (lamp voltage, filters, f/stop, field of views, binning) were used for all images, and fluorescence emission was normalized to photons per second per centimeter squared per steradian (p/s/cm2/sr). Images were acquired and analyzed using Living Image 2.5 software (Xenogen). Hairless SKH-1 mice on a chlorophyll-free diet (AIN-93G Purified Diet, Harlan Teklad, Madison, Wis.) for 1 week were administered intramuscularly 0.3 mg of biotin-cMORF25 microspheres in 100 μl PBS in the right thigh (target site) and an identical weight of native microspheres without biotin-cMORF25 in the left thigh as control. Within 30 minutes thereafter, all animals received either 1.0 nmol of Cy5.5-MORF25/BHQ3-cDNA18 or 1.0 nmol of Cy5.5-MORF25 in 100 μl by tail vein. In groups of three, animals were anesthetized and were imaged at various time points. All images were acquired using 1 s exposure time (f/stop 4). Regions of interest (ROI) of same size were centered about the microsphere injection sites of each thigh. The target/nontarget (i.e., study thigh to contralateral control thigh) ratio was considered a measure of the ability of the duplex to dissociate at its target in vivo.

Statistical analysis was performed using the Student's t-test with statistical significance assigned for P values less than 0.05. The mean fluorescence intensities of the target right thigh and of the contralateral left thigh were calculated using the region-of interest (ROI) function of Living Image software with background correction (Ke, S., et al., Cancer Res 63:7870-7875, 2003; Moon, W. K., et al., Bioconjug Chem 14:539-545, 2003).

### Results

### Surface Plasmon Resonance

Surface plasmon resonance was used to measure the rate of dissociation of the Cy5.5-MORF25/BHQ3-cDNA18 duplex in the presence of cMORF25. The biotinylated cDNA18 was immobilized on the streptavidin SA chip and the MORF25 injected to measure the association of the duplex and its dissociation rate constants. At that point, the duplex was formed on the chip and the cMORF25 was then injected to measure the duplex dissociation rate constant in the presence of cMORF25.

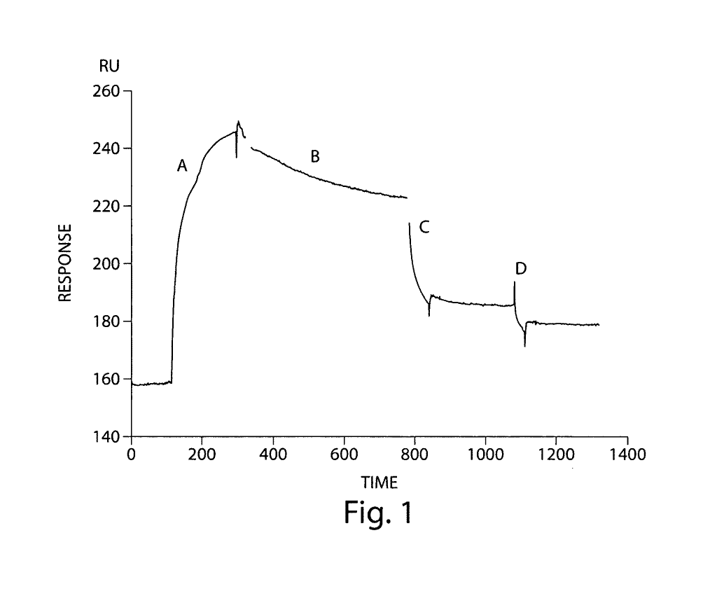

The sensorgram of FIG. 1 shows by the increase in response that the immobilized cDNA18 captured the flowing MORF25 (region A) to provide a duplex with a slow (region B) dissociation rate constant of 2.4×10⁻⁴ (1/s) shown by a slow decrease in response. Furthermore, when cMORF25 was allowed to flow over the now immobilized duplex, the cDNA18 was unable to prevent dissociation of the heteroduplex (region C) and the formation of a cMORF25YMORF25 homoduplex as shown by a decrease in response as the previously bound MORF25 is released as the duplex. The dissociation was very rapid with a rate constant of about 2.7×10⁻² (1/s) after the first injection of cMORF25 (region C) and 2.1×10⁻² (1/s) after a second injection of cMORF25 (region D).

### In Vitro Studies

Table 1 presented the fluorescence intensity under identical conditions of Cy5.5-MORF25 alone, BHQ3-cDNA18 alone and the Cy5.5-MORF25/BHQ3-cDNA18 duplex formed with a two- and a five-fold molar excess of BHQ3-cDNA18. More than 95% of the fluorescence of Cy5.5 was quenched even at the lowest molar ratio.

TABLE 1
The fluorescence intensity (F.I.) and the quenching
efficiency (Q.A.) in solution under identical conditions
of Cy5.5-MORF25 alone, BHQ3-cDNA18 alone, and the
Cy5.5MORF25/BHQ3-cDNA18 duplex formed with a two-
and a fivefold molar excess of BHQ3-cDNA18.

| Groups | Control 1 | Control 2 | Study 1 | Study 2 |
|---|---|---|---|---|
| Groups | Cy5.5- | BHQ3- | Cy5.5- | Cy5.5- |
| | MORF25 | cDNA18 | MORF25/ | MORF25/ |
| | alone | alone | BHQ3-cDNA | BHQ3-cDNA |
| | | | (1:2) | (1:5) |
| F.I. | 2475 ± 44 | −2 ± 2 | 140 ± 8 | 107 ± 2 |
| Q.E. | — | — | 95% | 96% |

As shown in Table 2, the identical fluorescence values for the study compared to control 1 confirms that the Cy5.5-MORF25/BHQ3-cDNA18 heteroduplex is dissociating in the presence of its cMORF immobilized target with binding of the Cy5.5-MORF25. The absence of fluorescence in control 3 is further confirmation of the binding. When the equal molar BHQ3-cDNA18 was added to the immobilized homoduplex, the fluorescent intensity increased only by about 30%, showing that the duplex is almost as efficient in binding to its target as a singlet.

TABLE 2
Fluorescence intensity (F.I.) under different conditions:
addition of the Cy5.5-MORF25/BHQ3-cDNA18 duplex to cMORF25
microspheres (study) and to native microspheres (control
3), the addition of the Cy5.5-MORF25/cDNA18 duplex (i.e.,
no BHQ3 inhibitor) to cMORF25 microspheres (control) and
the addition in sequence of Cy5.5-MORF25 singlet followed
by BHQ3-cDNA18 singlet (control 2).

| Groups | Study | Control 1 | Control 2 | Control 3 |
|---|---|---|---|---|
| Step 1 | cMORF25 | cMORF25 | cMORF25 | Native |
| | microspheres | microspheres | microspheres | microspheres |
| Step 2 | — | — | Cy5.5- | — |
| | | | MORF25 | |
| Step 3 | Cy5.5- | (Cy5.5- | BHQ3-cDNA | Cy5.5- |
| | MORF25/ | MORF25/ | | MORF25/ |
| | BHQ3- | cDNA18) | | BHQ3- |
| | cDNA18 | | | cDNA18 |
| F.I. | 665 ± 15 | 668 ± 14 | 894 ± 58 | 12 ± 1 |

### In Vivo Studies

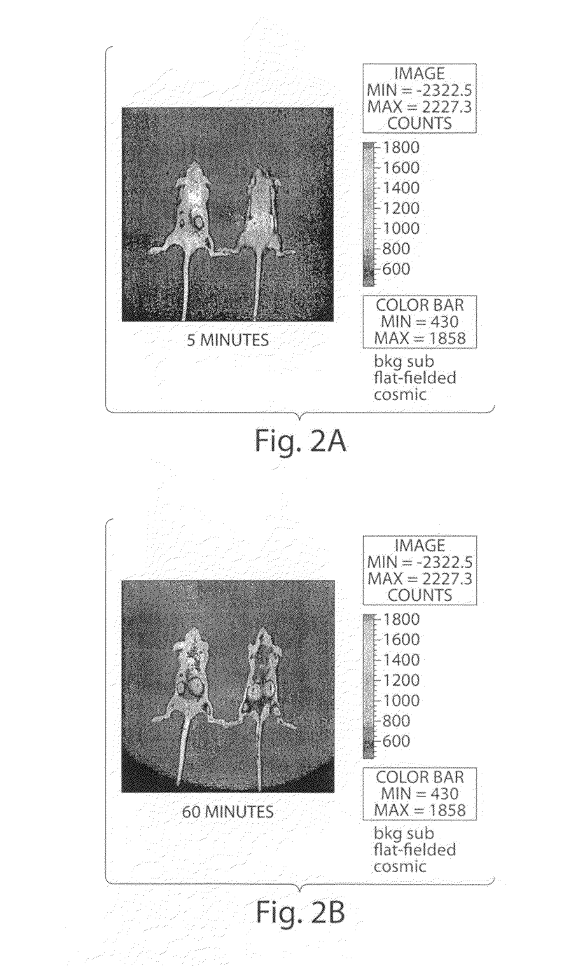

FIG. 2 presents the whole body fluorescence images obtained simultaneously of two hairless SKH-1 mice each implanted with microspheres in both thighs but with the cMORF25 target only on the microspheres in the right thigh of each animal. The animals received either the Cy5.5-MORF25/BHQ3-cDNA18 duplex (left mouse) or the Cy5.5-MORF25 singlet (right mouse) and were imaged simultaneously and repeatedly starting at 5 minutes. At 5 minutes (left panel), the background fluorescence in the animal receiving the Cy5.5-MORF25 is obviously high in kidneys and most normal tissue such that binding to its target in the right thigh is obscured. By contrast, the target thigh is clearly visible in the animal receiving the duplex. However, the images appear more similar at 60 minutes (right panel) because in the animal receiving the singlet, excess Cy5.5-MORF25 not bound to the target clears rapidly resulting in a decreasing background and possibly also because in the animal receiving the duplex, the excess duplex not bound to the target is dissociating resulting in an increasing background. That the fluorescence from the target thigh at 60 minutes is higher in animals receiving the singlet compared to the duplex may be due to differences in pharmacokinetic and accessibility but may possibly also reflect the somewhat higher efficiency of binding to immobilized cMORF25 of the singlet compared to the duplex shown in Table 2.

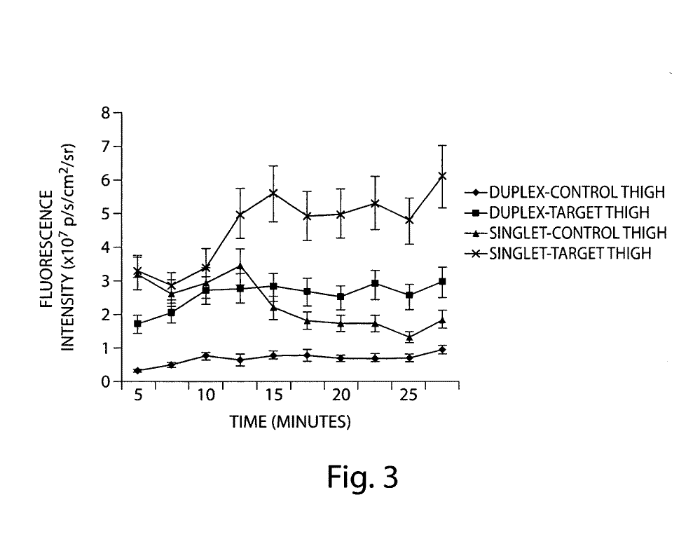

The results of repeat measurements of fluorescence in ROIs about both thighs are presented in FIG. 3 and confirm the persistence of fluorescence in vivo over 30 minutes. Also shown is the higher fluorescence in the study compared to control thighs in animals receiving either the duplex or the singlet.

### Discussion

An alternative technique to radioactivity imaging gaining in prominence is optical (fluorescence and bioluminescence) imaging. Optical imaging is already capable of surface imaging in patients of skin cancers, exposed tissues, and tissues accessible by endoscopy (Chen, C. S. et al., Br J Dermatol 153:1031-1036, 2005). Optical and radioactivity imaging share the potential for both high resolution and high sensitivity imaging (Houston, J. P. et al., J Biomed Opt 10:054010, 2005; Bloch, S. et al., J Biomed Opt 10:054003, 2005). While radioactivity imaging (particularly single photon imaging) can claim the largest number of contrast agents among the molecular imaging modalities, optical imaging may be second in this category. Optical imaging is also the least expensive of these modalities. Among the disadvantages, apart from an inability, at present, to quantitate contrast agent concentrations, optical imaging is limited in the living subject to millimeter-scale resolution due to light scatter and attenuation in deep tissues.

A major advantage of optical imaging over radioactivity imaging is the possibility of limiting signal expression only to the target itself. While radioactivity imaging, by its nature, requires that the contrast agent be administered while “active” (in this case radioactive), it follows that contrast agent not localized in the target will also be detected, often resulting in unfavorable target/nontarget radioactivity ratios. Optical imaging offers the important opportunity of administering contrast agents that are inactive and become active only in the target (McIntyre, J. O. et al., Biochem J 377:617-628, 2004). The most common examples involve “beacons” in which a DNA is constructed with a hairpin configuration bringing together two fluorophores at its ends. By being forced into close proximity, fluorescent emission of the fluorophores is inhibited. However, if the hairpin is opened up, the two fluorophores become separated, and thus, fluorescent (Zheleznaya, L. A., et al, Anal Biochem 348:123-126, 2006). Studies have been done to investigate an approach using linear duplexes rather than hairpin oligomers (Molenaar, C. et al., Nucleic Acids Res 29:E89-E89, 2001; Tyagi, S. et al., Biophys J 87: 4153-4162, 2004). In addition to pretargeting applications, another in vivo use under investigation in this laboratory is antisense optical imaging using an emitter-inhibitor DNA duplex designed to dissociate at the site of a target mRNA such that the antisense DNA within the duplex and conjugated with the emitter will bind, thereby, releasing the inhibitor and fluorescence inhibition only in the target.

The successful duplex for pretargeting applications must meet certain requirements. First, the emitter and inhibitor fluorophores must be so arranged that fluorescence is effectively inhibited in its duplex form. Secondly, the duplex must be sufficiently stable in vivo to survive intact everywhere but in its target. Lastly, the duplex must be so arranged that it effectively and rapidly dissociates at the site of its target. The duplex selected for this investigation was designed based on the results of earlier studies from this laboratory showing that MORF duplexes longer than 18 mer are stable in vitro and in vivo (He, J. et al., Bioconjug Chem 14:1018-1023, 2003; Liu, G. et al., Eur J Nucl Med 31:417-424, 2004) and that a MORF-DNA duplex is more stable than a DNA-DNA duplex with the same sequences (Zhang, Y. et al., Nucl Med Common 25:1113-1118, 2004). As the melting temperature of the 18 mer DNA-DNA duplex is calculated to be above 50° C., so must be the MORF-DNA duplex. In this way, the MORF25/cDNA18 duplex was selected for this proof of concept study and confirmed using SPR by measuring the dissociation rate constants and the ability of the cMORF25 to dissociate the immobilized MORF25/cDNA18 duplex (FIG. 1).

Subsequent studies used the fluorophore conjugated oligomers in vitro and in vivo. The near infrared fluorescent dye Cy5.5 has been used as a contrast agent for the in vivo demarcation of tumors by several groups (Ke, S. et al., Cancer Res 63:7870-7875, 2003; Ballou, B. et al., Cancer Detect Prey 22:251-257, 1998; Weissleder, R. et al., Nat Biotechnol 17:375-378, 1999; Petrovsky, A. et al., Chancer Res 63:1936-1942, 2003). This dye has an absorbance maximum at 675 nm and emission maximum at 694 nm and can be detected in vivo at subnanomole quantities and at tissue depths sufficient for experimental or clinical imaging depending on the fluorescent image acquisition technique. The BHQ-3 inhibitor has a maximum absorption in the 620 to 730 nm range which provides excellent quenching inhibition of Cy5.5 as was demonstrated in vitro (Table 1).

In this investigation, the Cy5.5 emitter was conjugated to the 3′ equivalent end of a MORF rather than DNA as this DNA analogue is reported to be stable in vivo for extended periods (He, J. et al., Bioconjug Chem 14:1018-1023, 2003). The BHQ3 inhibitor was conjugated to the 5′ end of a phosphorothioate DNA also known to be stable in vivo and has been shown to form a duplex with the intermediate stability required for this application (Zhang, Y. et al., Nucl Med Common 25:1113-1118, 2004). The relative in vitro stability and dissociation of this duplex has now been demonstrated in vitro and in vivo.

A microsphere mouse model rather than the more physiological xenograft mouse model was selected for this proof of concept optical pretargeting study, in part because microspheres provide complete control over the expression of the cMORF target, allowing direct comparison in contralateral thighs in the same animal of fluorescence in thighs with and without the cMORF target. The ratio of fluorescence in the target thigh compared to the contralateral control thigh then measures accurately the binding of the Cy5.5-MORF25 to its target, and in the case of the duplex, its dissociation therein. As shown in FIGS. 2 and 3, while no difference in this ratio is apparent at early times in mice administered the Cy5.5-MORF25 singlet, this ratio and the resulting image contrast are remarkably positive as early as 5 minutes post administration of the duplex. This positive difference between target thigh and control thigh proves that specific binding of the duplex occurred to its target and illustrates the potential advantages of using quenched duplexes over singlets for optical pretargeting. Additional studies will be required to optimize the duplex for this application.

The results described herein demonstrate that by using linear oligomer for optical pretargeting, chain lengths (and base sequences) may be manipulated to provide duplexes with stabilities and fluorescence inhibition optimized for pretargeting and other applications of optical imaging. This study of optical pretargeting provides a proof of concept that oligomer pretargeting found to be useful with radioactivity detection is applicable with fluorescent detection as well.

### Example 2

### Antisense Optical Imaging

### Introduction

Antisense targeting of tumor with fluorescent conjugated DNA oligomers has the potential of improving tumor/normal tissue ratios over that achievable by nuclear antisense imaging. When administered as a linear duplex of two fluorophore-conjugated oligomers arranged in a manner that inhibits fluorescence as the duplex and designed to dissociate only in the presence of the target mRNA, the fluorescence signal should in principle be inhibited everywhere except in the target cell. Optical imaging by fluorescence quenching using linear fluorophore-conjugated oligomers has not been extensively investigated and may not have been previously considered for antisense targeting.

Studies were performed to evaluate in cell culture and in KB-G2 tumor bearing nude mice a 25-mer phosphorothioate (PS) anti-mdr1 antisense DNA conjugated with the Cy5.5 emitter on its 3′ equivalent end and complexed as a linear duplex with a shorter 18-mer phosphodiester (PO) complementary DNA (cDNA) with the Black Hole-inhibitor BHQ3 on its 5′ end. In serum environments, 90% of the DNA25-Cy5.5 fluorescence was inhibited immediately following addition of the cDNA18-BHQ3 and showed only slight loss of inhibition over 24 h at 37° C. As evidence of specific binding, when incubated with the study DNA25-Cy5.5/cDNA18-BHQ3 duplex, the fluorescence was lower in KB-31 (Pgp+/−) cells compared to KB-G2 (Pgp++) cells, but when incubated with the control cDNA 18-Cy5.5/DNA25-BHQ3 duplex in which the fluorophores are reversed, the fluorescence of the two cell types were both low. As further evidence of specific binding, the fluorescent intensity of total RNA from KB-G2 cells incubated with the study duplex showed evidence of dissociation and hybridization with the target mRNA. Furthermore, the fluorescence microscopy images of KB-G2 cells incubated with DNA25-Cy5.5 as the singlet or study duplex show that migration in both cases is to the nucleus. The animal studies were performed in mice bearing KB-G2 tumor in one thigh and receiving IV the study or control duplexes. The tumor/normal thigh fluorescence ratio was clearly positive as early as 30 min postinjection in the study mice and reached a maximum at 5 h. By contrast, much lower fluorescence was observed in mice receiving the control duplex at the same dosage. Fluorescence microscope imaging showed that the Cy5.5 fluorescence was much higher in tumor sections from animal that had received the study rather than control duplex. Combining a fluorophore-conjugated antisense DNA with a inhibitor-conjugated shorter complementary cDNA inhibited fluorescence both in cell culture and in tumored animals except in the presence of the target mRNA. This proof of concept investigation demonstrates optical antisense targeting.

### Background

Hybridization properties of oligomers may have applications in optical imaging as an alternative to nuclear imaging. While nuclear imaging offers many advantages over other imaging modalities including high sensitivity of detection, quantitation accuracy and the availability of numerous contrast agents (Hnatowich, D. J., J Cell Biochem Suppl.; 39: 18-24, 2002), one disadvantage is the physics of nuclear decay that does not permit manipulation of the gamma rays emissions. A property of optical imaging unique among most molecular imaging modalities is the potential to modulate the detectable signal in the target. By bringing into close proximity two fluorophores, it is possible to either shift the emission fluorescence to a lower energy, higher frequency or to quench the fluorescence entirely by fluorescence resonance energy transfer (FRET) (Marras, S. A. et al., Nucleic Acids Res.; 30: e122, 2002). Molecular beacons consisting of DNAs in the form of hairpins with fluorophores on each end are already in use in this manner for in vitro assays and for vivo studies as well (Tyagi, S. et al., Nat. Biotechnol.; 14: 303-308, 1996; Silverman, A. P. et al., Trends Biotechnol.; 23, 225-230, 2005; Nitin, N. et al., Nucleic Acids Res.; 32:e58, 2004; McIntyre, J. O. et al., Biochem J.; 377:617-628, 2004; Hoeflich, K. P. et al., Cancer Res.; 66:999-1006, 2006).

Thus optical antisense targeting of tumor with fluorescent conjugated DNA oligomers may have the potential of improving tumor/normal tissue ratios. When administered as a linear duplex designed to inhibit fluorescence and to dissociate only in the presence of the target mRNA, the fluorescence signal in principle should be inhibited everywhere except in the target cell. Optical imaging by fluorescence quenching using linear fluorophore-conjugated oligomers has not been extensively investigated and may not have been previously considered for antisense targeting. In this proof of concept study, the target was again the mdr1 mRNA. It has now been demonstrated that mdr1 antisense DNA accumulates in the KB-G2 Pgp++ cells and to a lesser extent in the KB-31 Pgp+/− cells by an antisense mechanism (Nakamura, K. et al., J Nucl Med.; 46:509-513, 2005; Liu, X. et al, J Nucl Med.; 47: 360-368, 2006).

For optical antisense targeting, the properties of the duplex are exacting-the duplex must be sufficiently stable to remain intact in circulation and in normal tissues but yet sufficiently unstable to dissociate in the presence of the target mRNA. These studies have confirmed another property of the duplex, namely that it crosses cell membranes without entrapment (Liu, X. et al., Mol Imaging Biol. 2006 (Epub ahead of print)). This study has evaluated a strategy of delivering a 25-mer phosphorothioate (PS) mdr1 antisense DNA25 conjugated as a linear duplex with the Cy5.5 emitter on its 3′ equivalent end and complexed with a complementary shorter 18-mer phosphodiester (PO) mdr1 sense DNA18 with the BHQ3 inhibitor on its 5′ end. The DNA25-emitter and cDNA 18-inhibitor are so arranged that fluorescence is effectively inhibited with the DNAs in their duplex form. Once in the vicinity of its mRNA target, a new and stable antisense DNA25-Cy5.5/target mRNA duplex forms spontaneously with the release of the shorter DNA along with its inhibitor. This study was conducted in cell culture and in KB-G2 tumor bearing mice.

### Methods

The duplex consisted of an mdr1 uniform PS antisense DNA25 (5′-AAG-ATC-CAT-CCC-GAC-CTC-GCG-CTC-C (SEQ ID NO:3)) and a uniform PO complementary sense cDNA18 (5′-GGA-GCG-CGA-GGT-CGG-GAT (SEQ ID NO:4)). The antisense DNA sequence was elongated from the antisense 20 mer DNA used in these laboratories previously (underlined) (Nakamura, K., et al., J Nucl Med.; 46:509-513, 2005.). All DNAs were purchased HPLC purified, the antisense DNAs were obtained with Cy5.5 (Sigma-Proligo, Woodlands, Tex.) or Black Hole Quencher 3 (BHQ3, Biosearch Technologies, Novato, Calif.) on the 3′ end (designated herein as DNA25-Cy5.5 and DNA25-BHQ3) and the complementary sense cDNAs were obtained with Cy5.5 or BHQ3 on the 5′ end (designated herein as cDNA18-Cy5.5 and cDNA18-BHQ3). Regardless of whether the DNA was native (i.e without fluorophore) or whether the attached fluorophore was the Cy5.5 or BHQ3 and regardless of whether the DNA was used as the singlet or duplex, the antisense DNA was always the 25 mer and the sense complementary DNA was always the 18 mer. The KB-G2 (Pgp++) and KB-31 (Pgp+/−) human epidermoid carcinoma cell lines were a gift from Isamu Sugawara (Research Institute of Tuberculosis, Tokyo, Japan), and were cultured in Dulbecco's modified Eagle's media (DMEM) (Gibco BRL Products, Gaithersburg, Md.) containing 10% fetal bovine serum (FBS) for 18-24 h at 37° C., 5% CO₂. Cells were cultured until reaching about 60-70% confluence. The cells were incubated with DNAs in serum-free DMEM in the cell studies of this investigation.

### Duplex Preparation and Stability

Both the study DNA25-Cy5.5/cDNA18-BHQ3 duplex and the control cDNA18-Cy5.5/DNA25-BHQ3 duplexes were prepared by mixing the antisense DNA25 and complementary cDNA18 at a 1:1 molar ratio in 150 mM phosphate-buffered saline (PBS). The duplex mixtures were vortexed for 10 s, heated in a boiling water bath for 10 min to dissociate any intrastrand duplexes and allowed to cool to room temperature (25° C.).

To measure stability, the duplexes were added in triplicate at 100 μl per well to a 96-well plate to a final concentration of 0.1 nM in 10% FBS/DMEM and incubated at 37° C. for different times up to 24 h. The control was the singlet free DNA25-Cy5.5 added at the identical molar concentration in the identical manner. The fluorescence intensity (excitation 681 nm/emission 700 nm) was read at each time point on a SpectraMax M5/M5^{e} Microplate fluorescence plate reader (Molecular Devices Corporation, Sunnyvale, Calif.).

### Cell Studies

Rhodamine 123 (Sigma, St. Louis, Mo.) was used to confirm that the difference in multidrug resistance (i.e., Pgp expression) between the KB-G2 and KB-31 cells was preserved. Both cells were treated with the study duplex to a final concentration of 0.3 and, after 3 h, the cells were trypsinized and suspended in 1% FBS/DMEM. The cells were washed twice and resuspended in the same medium at 37° C. before adding Rhodamine 123 at 1 μg/ml. After 1 h at 37° C., the cells were washed twice and resuspended in ice-cold PBS buffer with 1% BSA. Viable cells were analyzed for the accumulation of fluorescence on a Becton Dickinson flow cytometer (BD Biosciences, Franklin Lakes, N.J.) using CellQuest software (BD Biosciences).

Two duplex accumulation studies were performed in KB-G2 and KB-31 cells in triplicate in 96-well black plates (Corning Inc., Corning, N.Y.) seeded at 5,000 cells per well and incubated with 10% FBS/DMEM culture medium overnight. In one study, the DNA25-Cy5.5/cDNA18-BHQ3 duplex and singlet DNA25-Cy5.5 were incubated with KB-G2 cells in serum-free DMEM at dosages of 0.02, 0.1 and 0.3 nmol for 3 h at 37° C. In the other study, the DNA25-Cy5.5/cDNA18-BHQ3 duplex and the control cDNA18-Cy5.5/DNA25-BHQ3 duplex were incubated with both KB-G2 and KB-31 cells in serum-free DMEM at the same dosage of 0.1 nmol for 3 h at 37° C. The cells were then washed twice with 10% FBS/DMEM and incubated for 1 h in 10% FBS/DMEM before being washed twice with PBS. The lysis buffer (10 mM Tris-HCl, pH 8.0, 150 mM NaCl, 1% Triton X-100) was then added at 100 μl per well and the cells were incubated for 1 h at 37° C. The fluorescence intensity of each lysis solution was measured on the fluorescence plate reader. The data was normalized to the total protein content of each well as determined by the Bradford protein assay (Pierce, Rockford, Ill.).

To measure total RNA binding, KB-G2 cells were seeded in triplicate in 6-well plates at 10,000 cells per well and incubated with 10% FBS/DMEM culture medium overnight. Duplex DNAs were added into each well with serum-free DMEM at dosages of 0.9 nmol/well and the cells incubated for 3 h at 37° C. Total RNA was extracted using the RNeasy® Mini extraction kit (Qiagen, Valencia, Calif.). Each sample was then added to the 96-well plate and fluorescence intensity was measured as above.

The intracellular distribution of fluorescence was measured in KB-G2 cells seeded onto glass bottom culture dishes and incubated in 10% FBS/DMEM overnight. The study duplex DNAs were added into each well in serum-free DMEM at a final concentration of 0.3 μM for 3 h at 37° C. The cells were then washed twice with 10% FBS/DMEM and incubated for 1 h in 10% FBS/DMEM before being washed twice with PBS. For living cell imaging, the cells were directly observed on a Nikon Eclipse TE 2000-S microscope (Nikon Instruments Inc., Melville N.Y.) equipped with Cy5.5 filter and CCD camera and the images processed using IPLab software (BD Biosciences). For nucleus colocalization analysis, the cells were further incubated with 1 μM Sytox Green (Molecular Probe, Eugene, Oreg.) in 95% ethanol for 15 min to fix and stain the nuclei before being washed twice with PBS and examined under the microscope equipped with filter (excitation 480 nm/emission 535 nm). In the microscopic images, Cy5.5 and Sytox Green were pseudocolored red and green respectively.

### Animal Studies

All animal studies were performed with the approval of the UMMS Institutional Animal Care and Use Committee. Male nude mice (NIH Swiss, Taconic Farms, Germantown, N.Y., 30-40 g) at 7 weeks of age were each injected subcutaneously in the right thigh with a 0.1 ml suspension containing 10⁶ KB-G2 cells with greater than 95% viability. Mice were used for imaging studies 14 days later when the tumors reached 0.4-0.6 cm in diameter and were placed on a chlorophyll-free diet (AIN-93G Purified Diet, Harlan Teklad, Madison, Wis.) for 5 days prior to imaging.

Mice bearing KB-G2 tumor were administered 3 nmol of the control duplex while additional animals received the study duplex at either 1, 3 or 5 nmol in 100 μl PBS per mouse via a tail vein. Mice were then anesthetized with i.p. ketamine (90 mg/kg) and xylazine (10 mg/kg). In vivo fluorescence imaging was performed on an IVIS 100 small animal imaging system (Xenogen, Alameda, Calif.) using a Cy5.5 filter set. Identical illumination settings (lamp voltage, filters, f/stop, field of view, binning) were used for all images and fluorescence emission was normalized to photons per second per centimeter squared per steradian (p/s/cm²/sr). Fluorescence images were acquired at 0.5, 3, 5 and 24 h after injection using 1 s exposure time (f/stop 4). Images and measurements of fluorescence signals were acquired and analyzed with Living Image 2.5 software (Xenogen, Alameda, Calif.). Regions of interest (ROI) of equal size were centered about the target sites and nontarget sites (i.e., control tumor, contralateral control thigh).

The tumors from mice administered either 3 nmol of the study duplex or the same dosage of control duplex were removed at 24 h, dissected and treated for fluorescent microscopy imaging. The tumors were placed in tissue holders that were then filled with Tissue-Tek Optimal Cutting Temperature Compound (Sakura Finetek USA, Torrance, Calif.) and immediately frozen in dry ice. The distribution of the fluorophore within tumor slices of 10 μm thickness was examined and images were obtained in the GFP channel to locate the cells and in the Cy5.5 channel to show targeting cells. The two images were merged.

### Statistical Analysis

Data are presented as the mean±S.D. of at least N=3 and statistical analysis was performed using Student's t-test. A p value <0.05 was considered statistically significant.

### Results

### Duplex Stability

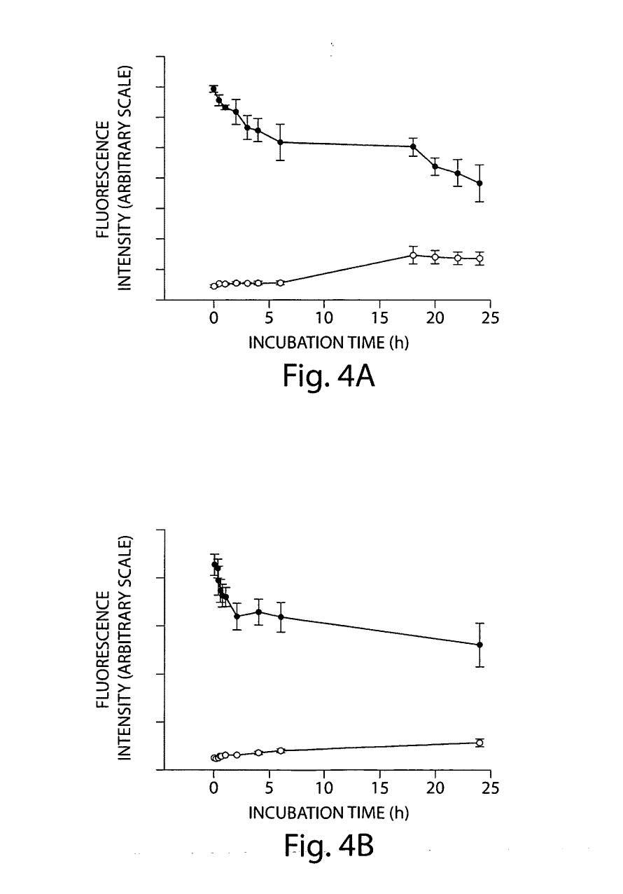

The stability of the DNA25-Cy5.5/cDNA18-BHQ3 duplex was evaluated in 37° C. in 10% FBS/DMEM (FIG. 4A) and 70% normal mouse serum (FIG. 4B) at 0.1 nM over 24 h and compared to the singlet DNA25-Cy5.5. The fluorescence intensities presented in FIG. 4 demonstrate that the duplex is sufficiently stable at least over 24 h in this medium. Compared to the fluorescence of the singlet, over 90% of the duplex fluorescence was quenched initially and largely remained so for about 6 h. Thereafter, the intensity increased but only slowly. The fluorescence intensities of the singlet decreased over this period presumably because of fluorescence decay (Eaton, D. F. et al., Pure & Appl Chem.; 62: 1631-1648, 1990)

### Cellular Studies

### Rhodamine 123 Accumulation

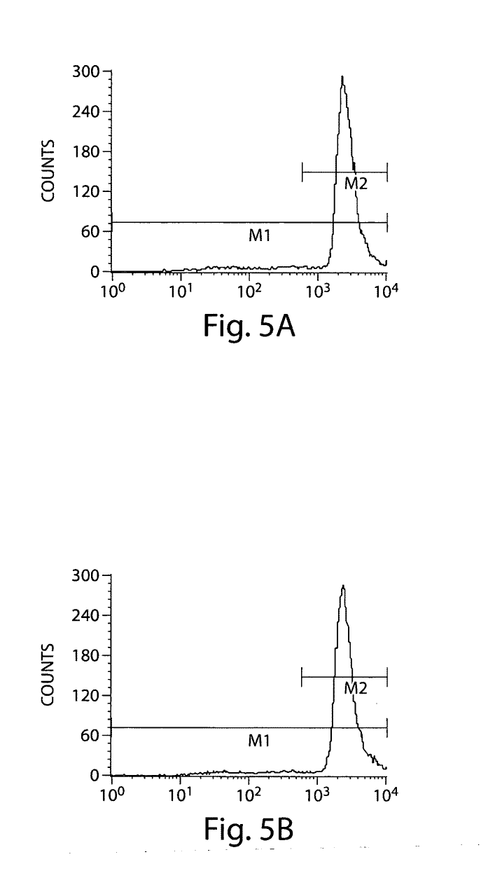

One major form of multidrug resistance to cancer therapeutic agents is mediated by overexpression of P glycoprotein (Pgp), a membrane protein ATPase that serves as a drug efflux pump (Alahari, S. K., et al., J. Pharm. Exp. Therapeutics.; 286: 419-428, 1998.). Rhodamine 123 is a substrate for the Pgp efflux pump and is often used as a surrogate for drug accumulations (Kang, H., et al., Nucleic Acids Res.; 32:4411-4419.18, 2004). The accumulation of Rhodamine 123 by KB-G2 (Pgp++) and KB-31 (Pgp+/−) cells were measured by flow cytometry after incubation with the study duplex at 0.3 μM. Fluorescence intensity was detected in the FL1-H channel. To obtain histogram from the FACS analysis, the gate M1 was placed to count all cells while the gate M2 was placed to count only those cells with high Rhodamine 123 fluorescence. Acquisition was set to 20,000 events and results depict the viable/intact cell population as routinely gated in the forward and side scatter plot. As shown in FIG. 5 before incubation with the study duplex, 93% of KB-31 cells showed high accumulations of Rhodamine 123 fluorescence (FIG. 5a) compared to only 53% of KB-G2 cells (FIG. 5c), no doubt because Rhodamine 123 was less effectively excreted from KB-31 cells because of a low Pgp expression level in these cells. Thus these results show that the KB-31 cells used in this investigation were low level expressors of Pgp compared to KB-G2. As also shown, incubation of KB-G2 cells with the duplex led to a dramatic 35% increase in Rhodamine 123 accumulation and therefore a strong inhibition of Pgp expression (FIG. 5d compared to FIG. 5c). By contrast, incubating KB-31 cells with the duplex had no effect (FIG. 5b compared to FIG. 5a). Thus these results also indicate that the duplex is capable of inhibiting Pgp expression by what is almost certainly an antisense effect.

### Cellular Accumulation

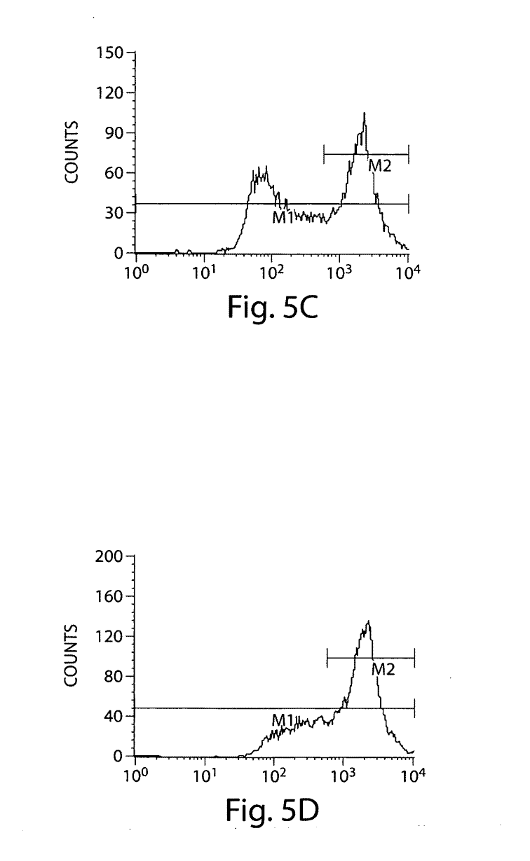

The KB-G2 cells were incubated with DNA25-Cy5.5 as the singlet and as the study DNA25-Cy5.5/cDNA18-BHQ3 duplex at 0.02 nmol, 0.1 nmol or 0.3 nmol for 3 h and fluorescence measured. The histograms of FIG. 6 (Panel A) show that the fluorescence intensity of the cells is in all cases significantly higher when incubated with the DNA25-Cy5.5 as the singlet compared to the duplex. The lower fluorescence of cell incubated with the duplex at the same molarity is therefore evidence that the DNA25-Cy5.5/cDNA18-BHQ3 duplex is still largely intact at 3 h, especially given that duplex DNAs appear to accumulate in cells more efficiently than singlet DNAs (Liu, X., et al., Mol Imaging Biol. 2006 (Epub ahead of print)).

Both the KB-G2 and KB-31 cells were incubated with both the study DNA25-Cy5.5/cDNA18-BHQ3 and the control cDNA18-Cy5.5/DNA25-BHQ3 duplex. As shown in FIG. 6 (Panel B), when incubated with the study duplex, the fluorescence was statistically lower (p=0.042) in the KB-31 cells compared to the KB-G2 cells as expected since the number of mRNA targets in KB-31 cells is limited. However, as also shown in the figure, the fluorescence intensities of the two cell types were statistically identical (p=0.211) when incubated with the control duplex, also as expected since in this case the Cy5.5 fluorophore has been placed on the sense DNA and therefore without affinity for the target mRNA. Both these results provide evidence of specific binding.

### RNA Binding

The KB-G2 cells were incubated with the DNA25-Cy5.5 as the singlet or study duplex at the same molar concentration for 3 h. Total RNA was then extracted and the fluorescence measured. The relative fluorescence intensities in total RNA for cells exposed to the duplex DNA was 189±61 (N=3) and therefore lower than in cells exposed to the singlet DNA at 544±289 (N=3). Under the reasonable assumption that the fluorescence of total RNA is due to specific binding of the DNA25-Cy5.5 to the target mRNA, the higher fluorescence intensity of total RNA incubated with the singlet DNA25-Cy5.5 is not surprising since the duplex requires dissociation before binding. That the RNA binding of the duplex was still about 35% of that of the singlet is therefore promising. The fluorescent intensities of the mRNA samples were too low to provide a meaningful result.

### Intracellular Distribution

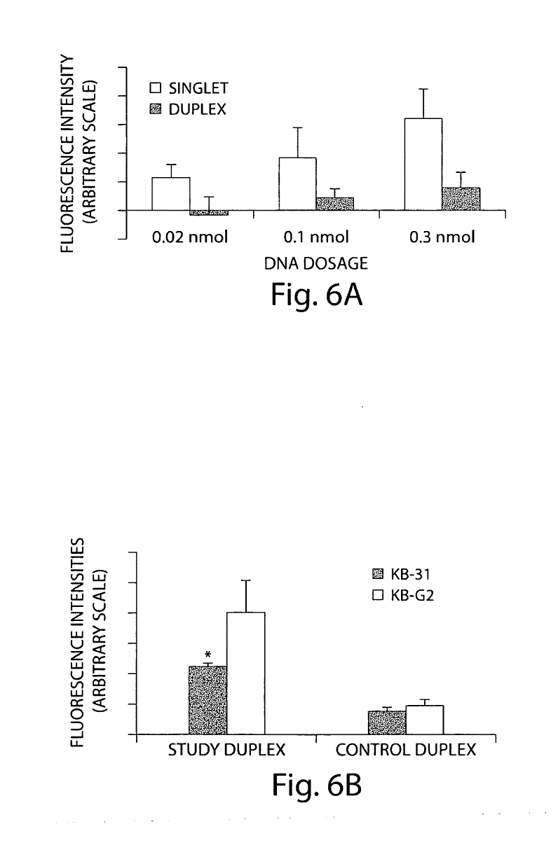

The fluorescence images of live and fixed KB-G2 cells are shown in FIG. 7 after incubation with the DNA25-Cy5.5 as the singlet or duplex. Live cells (Panel A) show similar fluorescence when incubated with the duplex compared to singlet but in both cases accumulations appear in the nucleus. That the intracellular distributions in fixed cells (Panel B) are identical for cells previously incubated with the singlet or duplex and that the accumulations is primarily in the nucleus is obvious by comparison of the Cy5.5 images with that of the nucleus staining Sytox Green. These results suggest that both forms of DNA25-Cy5.5 have the same cellular target and that the target is the nucleus.

### Animal Studies

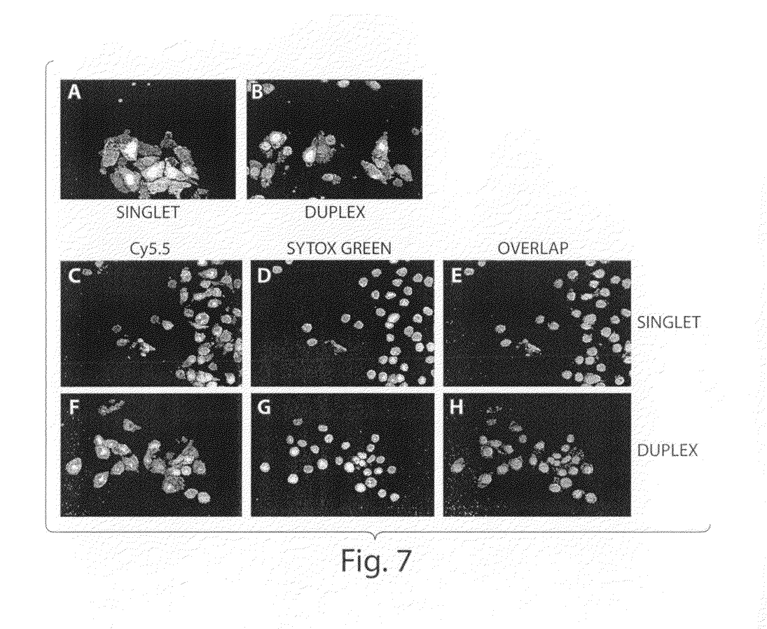

FIG. 8 presents whole body fluorescence images at 5 h in the dorsal view of mice with KB-G2 tumors and images of the sectioned excised tumors in animals receiving the study duplex (Panel A) or the control duplex (Panel B) at 3 nmol. The whole body image of the animal receiving the control duplex shows little or no Cy5.5 fluorescence. By contrast, images obtained simultaneously of the animal receiving the study duplex show pronounced whole body fluorescence and an obvious accumulation in the tumored thigh (Arrow). The fluorescence in the tumor thigh of mice increased with increasing dosage of study duplex (data not presented), however the background fluorescence also increased in proportion possibly because of instability of the study duplex towards dissociation in background tissues.

Further evidence for the preferential targeting of tumor by the study duplex is shown in the figure in the ex vivo images showing higher Cy5.5 fluorescence in tissues from the animal receiving the study duplex compared to the control duplex. The identical slices were also imaged in the GFP channel to locate the tumor cells within each section. As shown, the composite images clearly show that the Cy5.5 fluorescence (red) originates in the tumor cells (green).

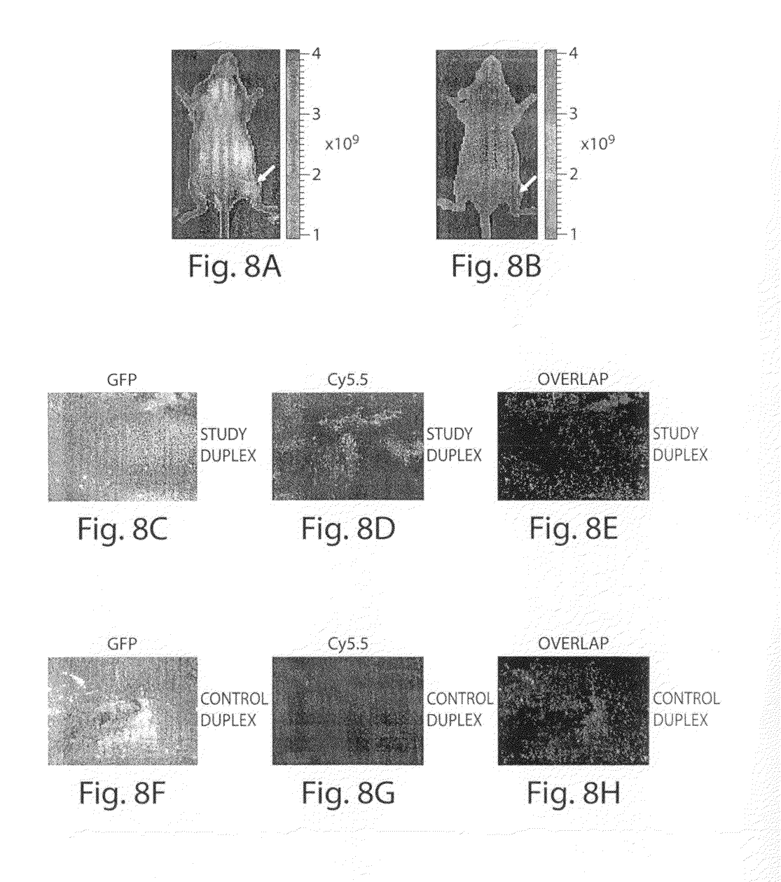

Regions of interest of equal area were placed over each animal's tumor and contralateral thigh and the fluorescent intensities ratios calculated for all four measurements over the 24 h imaging period. As shown in the histograms of FIG. 9, a positive tumor/normal thigh ratio was achieved as early as 30 min postinjection. At all time points, the tumor/normal ratio was never less than about 1.2 to 1.6 times higher in the animal receiving the study duplex compared to the animal receiving the control duplex.

### Discussion

Optical imaging has emerged as an attractive modality capable of investigating biological/molecular events in both cell culture and small living subjects. The modality is noninvasive, highly sensitive and affordable. Furthermore, the use of fluorescent contrast agents, unlike radiolabeled contrast agents, provides the potential of modulating the detectable signal in the target (Becker, A. et al., Nat. Biotechnol.; 19: 327-331, 2001; Cheng, Z., et al., Bioconjug Chem.; 17:662-669, 2006; Ntziachristos, V. et al., Eur Radiol.; 13: 195-208, 2003). A disadvantage to optical imaging is the much greater influence of tissue absorption and scatter on a fluorescent signal compared to a nuclear signal. Nevertheless, the use of fluorophores emitting in the near-infrared (700-900 nm) range minimizes tissue absorption and also minimizes autofluorescence from nontarget tissues (Massoud, T. F. et al., Genes Dev.; 17: 545-580, 2003). Many studies have now demonstrated that optical imaging is practical in small animals (Becker, A. et al., Nat. Biotechnol.; 19: 327-331, 2001; Cheng, Z. et al., Bioconjug Chem.; 17:662-669, 2006; Weissleder, R. et al., Nat. Biotechnol.; 17: 375-378, 1999)

These laboratories are investigating antisense DNAs and other oligomers and lately are investigating the use of double-stranded duplexes to improve cellular delivery (Liu, X., et al., Mol Imaging Biol. 2006 (Epub ahead of print)). We and others have compared single-stranded and double-stranded DNAs or siRNA and have shown that double-stranded oligomers (Liu, X., et al., Mol Imaging Biol. 2006 (Epub ahead of print); Jekerle, V. et al., J Pharm Pharm Sci.; 8: 516-527, 2005; Asriab-Fisher, A. et al., Biochem Pharmacol.; 68: 403-407, 2004) are stable both in cell culture and in animal study (Aharinejad, S. et al., Cancer Res.; 64: 5378-5384, 2004) and appear to show greater accumulates in cells compared to singlet oligomers (Liu, X., et al., Mol Imaging Biol. 2006 (Epub ahead of print)). However a duplex under consideration for antisense targeting must be sufficiently stable to remain intact in circulation and throughout its passage across the cell membrane but not so stable as to interfere with its dissociation and the subsequent binding of the now free labeled antisense DNA to its mRNA target.

While use of a radiolabeled duplex has now been shown to improve delivery of radiolabeled antisense oligomers, the free duplex in surrounding tissues provide an unwanted background radioactivity level. However, if a fluorescent label were used in place of the radioactivity label and if the complementary oligomer were to contain an inhibiting fluorophore, in principle free duplex would not fluoresce and would result in an improved target/nontarget ratio. This approach is similar to that already in practice in connection with stem-loop hairpin oligomers (Tyagi, S., et al., Nat. Biotechnol.; 14: 303-308, 1996; Bernacchi, S., et al., Nucleic Acids Res.; 29: E62-2, 2001). This report describes the first attempt to use linear duplexes for fluorescent quenching in connection with antisense targeting.

These laboratories and others have observed that the stability of the PS DNA/PO DNA heteroduplex was higher than an equivalent PS DNA/PS DNA homoduplex (Liu, X., et al., Mol Imaging Biol. 2006 (Epub ahead of print); Venkiteswaran, S. et al., Biochemistry.; 44: 303-312, 2005; Boczkowska, M. et al., Biochemistry; 41:12483-12487, 2002) and that duplexes consisting of two 25 mer phosphorodiamidate morpholino (MORF) oligomers show less evidence of instability in vitro and in vivo compared to duplexes with one or two 15 mer MORF oligomers (He, J. et al., Bioconj Chem.; 14: 1018-1023, 2003). For these reasons a 25 mer PS DNA25/18 mer PO cDNA18 duplex was selected for this study.

The identical antisense DNA sequence used previously in these laboratories (but elongated to 25 mer) and the mdr1 target mRNA was again used in this investigation. When radiolabeled, successful tumor targeting of this antisense PS DNA by an antisense mechanism was achieved in cell culture (Nakamura, K. et al., J Nucl Med.; 46:509-513, 2005) and in tumored mice (Nakamura, K., et al., Biocon Chem.; 15: 1475-14803, 2004). Furthermore, by targeting the mdr1 mRNA, the cells showing high level (KB-G2) and low level (KB-31) mdr1 mRNA and therefore different Pgp expression may again be used as a study/control pair. Since the mdr1 mRNA expression of the control Pgp+KB-31 cells can be variable and the cells can be inadvertently modified to express mdr1 mRNA and Pgp at levels approaching that of the study Pgp++KB-G2 cells (Nakamura, K. et al., J Nucl Med.; 46:509-513, 2005), Rhodamine 123 was used to confirm that the difference in multidrug resistance (i.e. Pgp expression) between the KB-31 and KB-G2 cells was preserved, as shown in FIG. 5.

The Cy5.5 emitter was selected because of its emission in the near infrared and BHQ3 was selected as a nonfluorescent quencher both because of its lack of self fluorescence and because it provides spectral overlap over the entire range of Cy5.5 fluorescence. Thus when the 3′-Cy5.5 conjugated antisense DNA is hybridized with the 5′-BHQ3 complementary cDNA, about 90% of the Cy5.5 fluorescence is quenched as shown in FIG. 4. As also shown in the figure, the duplex is stable in 10% serum over 24 h. Furthermore, as shown in FIG. 6A, when incubated with KB-G2 cells the fluorescence intensity of DNA25-Cy5.5 was much lower as the duplex compared to singlet at all dosages as evidence of duplex intracellular stability. To address the question of whether the fluorescence of the duplex is specific and the result of antisense targeting, the identical studies were performed with the Cy5.5 on the complementary sense strand and the BHQ3 on the antisense strand (i.e. cDNA18-Cy5.5/DNA25-BHQ3 duplex) and the results compared with the study (i.e. DNA25-Cy5.5/cDNA18-BHQ3) duplex after incubation in the KB-G2 cells with the high mdr1 mRNA level and KB-31 cells with a lower mdr1 mRNA level. As shown in FIG. 6B, the cellular accumulations of the control duplex was statistically identical in both cells while the accumulation of the study duplex was statistically higher in the KB-G2 cells as evidence of specific antisense binding.

Further evidence for specific antisense binding of the duplex was obtained in the fluorescence images of KB-G2 cells shown in FIG. 7. Evidence from this laboratory (Liu, X. et al. J Nucl Med.; 47: 360-368, 2006) and other laboratories (Alahari, S. K. et al., J. Pharm. Exp. Therapeutics; 286: 419-428, 1998; Jekerle, V. et al., J Pharm Pharm Sci.; 8: 516-527, 2005; Toub, N. et al., Oligonucleotides; 16: 158-168, 2006) show that antisense DNAs migrate to the nucleus of target cells. The images in the figure for both the live and fixed KB-G2 cells show similar migration of DNA25-Cy5.5 to the nucleus when incubated as the singlet or duplex. Finally, that incubation of KB-G2 cells with the study duplex increased the accumulation of Rhodamine 123 as shown in FIG. 5 may also be taken as evidence of specific targeting since the duplex in this case was apparently interfering with Pgp efflux pump expression and therefore mdr1 mRNA translation.

The whole body fluorescent images of KB-G2 tumor bearing mice presented in FIG. 8A provides further evidence in support of the hypothesis that the duplex dissociates as expected in the presence of its target mRNA and provides evidence that it does so in vivo at the tumor site. The figure presents images at 5 h of two identical tumored mice receiving either the study or control duplex and at the same 3 nmol dosage and imaged simultaneously. The increased fluorescence in the tumored thigh in the animal receiving the study duplex is apparent in these images as well as in the images obtained following administration of the 1 nmol and 5 nmol dosage of study duplex (data not presented). Regions of interest of equal area were set around both thighs in all animals to generate the ratio of fluorescence in the study thigh compared to normal thigh. As shown in FIG. 9, this ratio was never less than 1.2 to 1.6. Also presented in the FIG. 8 are fluorescent microscopy images of tumor slices taken from animals receiving either the study or control duplexes. By comparing the images with that obtained by imaging in the GFP channel, it is apparent that the Cy5.5 fluorescence is emanating from the tumor cells as evidence of tumor specificity.

An interesting observation within FIG. 8A is the background fluorescence in animals receiving the study but not the control duplex. Since more than 90% of the Cy5.5 fluorescence may be quenched in the duplex (FIG. 4), much lower backgrounds were expected. It was hypothesize that the study duplex (PS DNA25-Cy5.5/PO cDNA18-BHQ3 used in this investigation is unstable to dissociation in circulation and/or in normal organs within the 3 h period between administration and imaging. Thus once the PO DNA-BHQ3 in its singlet form is released, it is expected to rapidly degrade in vivo as any singlet phosphodiester DNA (Sands, H. et al., Mol. Pharmacol.; 45: 932-943, 1994). By contrast, the free phosphorothioate DNA-Cy5.5 will be stable to enzymatic degradation along with its fluorescence. However, when the control duplex dissociates, the PO DNA-Cy5.5 will be released to be degraded within minutes such that the Cy5.5 and its fluorescence may clear the cell and the whole body along with its fluorescence. To investigate this possibility, two tumored animals were imaged immediately following administration of a PO DNA-Cy5.5 or a PS DNA-Cy5.5. At 12 min, the mouse receiving the PO DNA-Cy5.5 showed minimal fluorescence in blood and, in this case, high florescence in kidneys and bladder. By contrast, the mouse receiving the PS DNA-Cy5.5 showed whole body fluorescence similar to that of FIG. 8A (data not presented).

These results indicate that fluorophore-conjugated linear DNA duplexes may be used to provide a fluorescent image of tumor in mice by an antisense mechanism. It has now been shown that selection of Cy5.5 and BHQ3 conjugated DNAs provides efficient fluorescent quenching when hybridized and florescence in tumor cells in culture and tumors in vivo when dissociated in the presence of the mRNA target.

### Example 3

### Comparison of Several Linear Fluorophore- and Quencher-Conjugated Oligomer Duplexes for Stability, Fluoresence Quenching, and Kinetics In Vitro and In Vivo

### Introduction

A useful property of optical imaging is the potential to modulate the detectable signal to improve target/nontarget ratios. When administered as a dimer of a fluorophore- and a quencher-conjugated duplex arranged to inhibit fluorescence but designed to dissociate only in the presence of its target, the fluorescence signal should in principle appear only in the target. The feasibility of this approach has been demonstrated by using a duplex consisting of a linear oligomer conjugated with Cy5.5 (emitter) hybridized to another linear oligomer conjugated with Iowa Black (quencher) in a pretargeting optical study. Now eight duplexes consisting of combinations of 18 mer linear phosphodiester (PO) and phosphorothioate (PS) DNAs and phosphorodiamidate morpholinos (MORFs) conjugated with Cy5.5 (emitter) and Iowa Black (quencher) were variously screened for in vitro duplex stability. The MORF/PO duplex was selected for further study based on evidence of stability in 37° C. serum. Simultaneously, the kinetics of quenching were investigated in vitro and in vivo in mice. Thereafter, mice were implanted in one thigh with MORF/PO Cy 5.5 microspheres and the complementary PS Iowa Black administered iv to measure the extent and kinetics of duplex formation in the target. While all duplexes were stable in buffer, only the MORF/PO duplexes and possibly all PS containing duplexes were stable in 37° C. serum for at least 4 h. The kinetics of quenching were found to be rapid in vitro, with a 80-90% decrease in Cy5.5 fluorescence immediately following formation of a PS/PS homoduplex, and in vivo, with a 27 to 38% decrease in target thigh/nontarget ratio within 1 h following administration of the complementary PS Iowa Black complementary DNA but not the random control DNA to mice implanted with MORF/PO Cy5.5 microspheres. This investigation has provided additional evidence that Cy5.5 may be efficiently and rapidly quenched by Iowa Black when both are conjugated to complementary oligomers and that the resulting inhibition of fluorescence is sufficiently persistent for imaging.

### Methods

All oligomers were either uniform PO, PS, or MORF throughout their length. All DNAs, fluorophore/quencher conjugated or native, were purchased HPLC purified (Integrated DNA Technologies, Inc., Coralville, Iowa) as were the MORFs, biotin conjugated, or native (GeneTools, Philomath, Oreg.). The DNAs were purchased with the fluorophore/quenchers attached directly while the biotin was attached to the MORF via a six-carbon linker. The oligomer base sequences of this investigation are shown in Table 3. For convenience, the 18 mer sequence was selected as the standard sequence of ongoing pretargeting studies (Liu, G., et al., Eur. J. Nucl. Med. Mol. Imaging. 31, 417-424, 2004). The calculated melting temperatures of PO homoduplexes with the 18 mer standard sequence and with the new sequence of this investigation are above 43° C. The melting temperatures of the PS and MORF duplexes of this investigation are also expected to exceed 37° C. whether native or with fluorophore/quencher attached (Zhang, Y. et al., Nucl. Med. Commun. 25, 1113-1116, 2004; Moreira, B. G. et al., Biophys. Res. Commun. 327, 473-48414, 2005). Complementary sequences are designated herein as cDNA or c′DNA and random sequences are designated as rDNAs. The base sequence of both the 36 mer PO and PS DNAs consisted of two complementary regions, an 18 mer sequence complementary to the above standard sequence (i.e., cDNA1) and an additional 18 mer new sequence (i.e., c′DNA2) complementary to that of a new 18 mer PS DNA2 sequence. Therefore, herein, both the 36 mer PO and PS DNAs are represented as cc′DNAs to illustrate this structure. The new sequence was selected to avoid hairpin formation and to bring the fluorophore/quencher into the proper configuration for quenching after hybridization (Scitools OligoAnalyzer 3.0, Integrated DNA Technologies). The control PS rDNA1 and PS rDNA2 were also 18 mer but with randomized sequences. The 1.0 m streptavidin-coated polystyrene microspheres had a 3.5 μg/mL biotin binding capacity (Polysciences, Inc., Warrington, Pa.).

All centrifuge tubes, pipet tips, and PBS buffers were autoclaved before use. The Cy5.5 fluorophore was selected because its emission maximum is at 694 nm and therefore in the near-infrared where light absorbance in tissue reaches a minimum. The Cy5.5 fluorescence can therefore be detected in vivo at subnanomolar concentrations and at depths sufficient for experimental studies in small animals and possibly in patients (Weissleder, R. e al., Nat. Biotechnol. 17, 375-378, 1999; Petrovsky, A. et al., Cancer Res. 63, 1936-1942, 2003; Ke, S. et al., Cancer Res. 63, 7870-7875, 2003; Chen, X. et al., Cancer Res. 64, 8009-8014, 2004). The Iowa Black RQ dye was selected because it has a broad absorbance spectrum ranging from 500 to 700 nm with peak absorbance at 656 nm and therefore that overlaps the emission spectrum of Cy5.5 such that the emissions of Cy5.5-DNA may be efficiently inhibited (quenched). The Cy5.5 fluorescence was detected using a 615-665 nm excitation filter combined with a 695-770 nm emission bandpass filter on a IVIS 100 optical camera (Xenogen, Alameda, Calif.). Regions of interest were set and data were analyzed with Living Image software (Xenogen). To reduce autofluorescence, the animals were hairless male SKH-1 (Charles River Breeding Labs, Wilmington, Mass.) and were fed on a chlorophyll-free diet (AIN-93G Purified Diet, Harlan Teklad, Madison, Wis.).

### Duplex Stability Studies by PAGE

All oligomers for the duplex stability studies were native rather than fluorophore/quencher conjugated and all were prepared at 10 μM concentration. The PAGE Ready Gel 15% TBE gel was purchased (Bio-Rad, Richmond, Calif.) and run in 1×TBE (Tris-borate buffer) at 87 v for 1 h then stained with ethidium bromide (0.5 mg/mL in 1×Tris-borate buffer). The following four duplexes were prepared: PO DNA1/PO cc′DNA; MORF1/PO cc′DNA; MORF1/PS cc′DNA; and PO DNA1/PS cc′DNA. All duplexes were first formed by heating at 90° C. for 10 min before cooling to room temperature. Samples were then incubated in PBS at 37° C. for 2, 4, or 9 h before analysis. For analysis, 1 μL was added to each well. Samples were also incubated in fresh mouse serum by adding 10 μL of each sample to 30 μL of serum followed by incubation at 37° C. for 2 or 4 h before analysis. For analysis, 4 μL was added to each well except for serum alone in which 3 μL was added.

### Confirmation of Fluorescence Hybridization Quenching in Vitro

The 18 mer PS Cy5.5 cDNA1 was first added in triplicate to Costar 96 well black polystyrene flat bottom assay plates (Corning Incorporated, Corning, N.Y.) at concentrations of 1, 2, or 5 μM in room-temperature 50 μL PBS (pH 7.4) ([Na⁺]=150 mM, pH 7.4) or 75% fresh mouse serum followed immediately by the addition of the 18 mer PS Iowa Black DNA1 to the wells at different cDNA1:DNA1 molar ratios. Fluorescence intensity was measured over 1.5 h thereafter on the IVIS camera. As control, the identical study was repeated with the random control PS Cy5.5 rDNA1 in place of the study PS Cy5.5 cDNA1. The kinetics of quenching was also measured in 75% fresh mouse serum in wells containing 5 μM PS Cy5.5 cDNA1 to which the PS Iowa Black DNA1 was added at a 1:1 molar ratio.

Before performing imaging studies on animals implanted with microspheres, the ability of PS Iowa Black DNA2 to quench the fluorescence of PO Cy5.5 cc′DNA immobilized on microspheres was established. Streptavidin-coated microspheres (100 μL) were added to a solution of 0.8 nmol of the duplex formed previously between biotin-MORF1 and PO cc′DNA and incubated at room temperature for 30 min. Thereafter the microspheres were separated by centrifugation and washed in PBS/BSA binding buffer three times.

Approximately 20 μL of these microspheres (Cy5.5 concentration 2 μM) were added to each well of the flat bottom assay plate and suspended in 50 μL of PBS/BSA binding buffer. Thereafter, the PS Iowa Black DNA2 was added at different cc′DNA:DNA2 molar ratios followed by agitation to suspend the microspheres. The fluorescence intensity was measured at room-temperature 20 and 60 min later on the IVIS camera, and the results were analyzed by Living Image software as before.

### Animal Studies

All animal studies were performed with the approval of the UMMS Institutional Animal Care and Use Committee. For in vivo studies, the identical illumination settings (lamp voltage, filters) were used for all images, and fluorescence emission was normalized to photons per second per centimeter squared per steradian (p/s/cm2/sr). All data were processed by Living Image 2.50 software (Xenogen). Prior to performing the imaging studies on microsphere implanted animals, the stoichiometry and kinetics of fluorescence hybridization quenching was studied in vivo. In groups of 3 to 4, male SKH-1 mice were injected via a tail vein with either 0.3 nmol (2 μg) or 0.8 nmol (5 μg) of the PS Cy5.5 cDNA1 in 0.1 mL, and thereafter each received via a tail vein either 0.3 nmol, 0.8 nmol or 2.4 nmol of PS Iowa Black DNA1. The molar ratios of Iowa Black DNA1 to Cy5.5 cDNA1 were either 1:1 or 3:1. The animals were then anesthetized using ketamine (90 mg/kg) and xylazine (10 mg/kg) and imaged on the IVIS camera both in the ventral and dorsal views at various times postinjection while under continuous sedation. All images were acquired with 1 s exposure (f/stop=2). A large region of interest was set about the whole animal for quantitation.

Approximately 30 μL of the microspheres prepared as described and carrying 0.06 nmol of Cy5.5 were implanted intramuscularly in one thigh of each SKH-1 mice. After 15 min, each animal received either 0.06 nmol (N=2), 0.6 nmol (N=2) or 6.0 nmol (N=4) of the PS Iowa Black DNA2 by iv administration and was imaged in both the dorsal and ventral view immediately thereafter. The fluorescence intensity in a region of interest about both thighs was recorded, and target to nontarget ratios were compared before and after the administration of PS Iowa Black DNA2 with any decrease defined as the quenching efficiency. As a control, one implanted animal received 6.0 nmol of the random Iowa Black rDNA2.

Although the amount of microspheres implanted was identical, the depth of intramuscular implantation varied from animal to animal and resulted in differences in the fluorescence intensity. To correct for absorption and scatter, the fluorescence intensity of all mice but one with implanted PO Cy5.5 cc′DNA microspheres were measured before the PS Iowa Black DNA2 was administrated. The remaining mouse left as a further control did not receive PS Iowa Black DNA2.

### Results

Reported herein is the screening of duplexes consisting of phosphodiester (PO) and phosphorothioate (PS) DNAs and/or phosphodiamidate morpholinos (MORFs) conjugated with Cy5.5 as emitter and Iowa Black as quencher to evaluate duplex stability in vitro and in vivo and to measure the pharmacokinetic and optical imaging properties of a stable duplex in a mouse microsphere model. The results of these measurements will be useful for selecting duplexes for future investigations.

### Duplex Stability Studies by PAGE

All four duplexes: PO DNA1/PO cc′DNA; MORF1/PO cc′DNA; MORF1/PS cc′DNA; and PO DNA1/PS cc′DNA, were found to be stable in 37° C. PBS. In each case, one intense band at about 50 base pairs was evident in PAGE gels for both homoduplexes and the MORF1/PS cc′DNA heteroduplex. Two weaker bands in the case of the MORF1/PO cc′DNA heteroduplex appeared consistently in all gels.

However, duplex stability in serum compared to PBS was very different. Of the four duplexes, only the MORF1/PO cc′DNA duplex showed evidence of stability at the earliest time point at 2 h. This band was weaker although still readily apparent at 4 h. The 9 h gels were equivocal as regards duplex stability. Weak bands appearing at low molecular weight in the case of the PO DNA1/PO cc′DNA were probably the result of the nuclease instability of this DNA. Finally, a band appeared at the loading point in all serum samples, including that of serum alone. However, this band was much more intense in the case of the PS cc′DNA duplexes presumably because of the protein binding affinity of this DNA. This analysis therefore cannot exclude the possibility that the duplexes with PS cc′DNA were also stable in serum but remained at the loading point.

An additional four 18 mer duplexes were also investigated in buffer and in serum: MORF1/PO cDNA1; MORF1/PS cDNA1; PO DNA1/PO cDNA1; PO DNA1/PS cDNA1. The results showed once again that at 2 and 4 h of incubation, the MORF1/PO cDNA is stable in serum.

It is possible to conclude from these duplex stability studies that the PO DNA homoduplex with overhang is unstable as expected due to the recognized instability to nucleases of a single chain PO DNAs (Tidd, D. et al., Br. J. Cancer 60, 343-350, 1989; Shaw, J. P. et al., Nucleic Acids Res. 19, 747-75020, 1991). However nuclease attack on PO DNA, including the single chain overhang, appears to be less effective when hybridized to MORF. The bead studies described below require streptavidin microspheres to which Cy5.5-conjugated oligomer are bound. Since neither a PO or PS DNA derivitized with both Cy5.5 and a biotin group on opposite ends were commercially available, the results of the above PAGE study provided confidence that microspheres prepared by the addition of biotinylated MORF1 first to the beads followed by addition of PO cc′DNA-conjugated Cy5.5 would be stable.

### Confirmation of Fluorescence Hybridization Quenching in Vitro

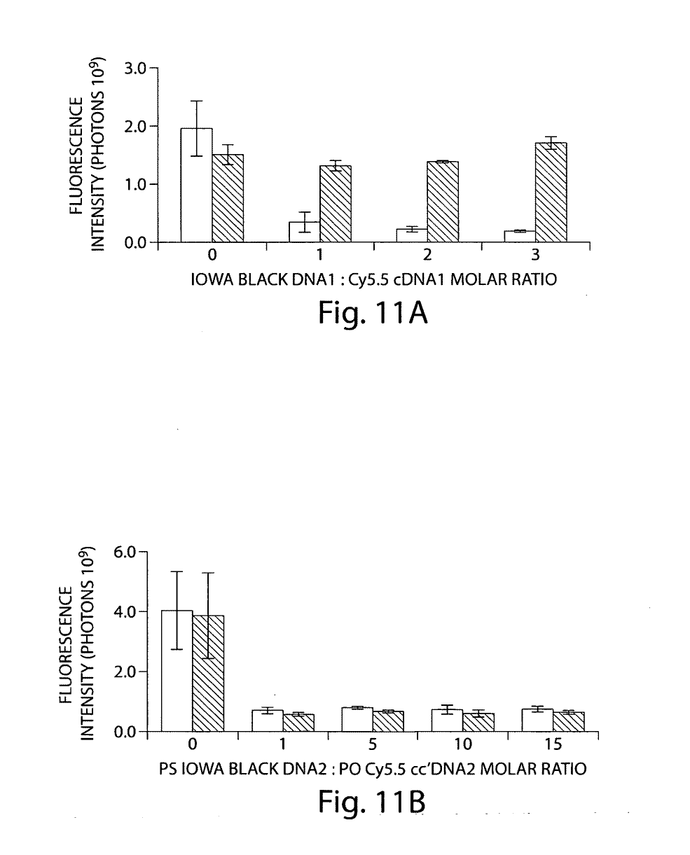

The histograms of FIG. 11A show that the fluorescence in PBS buffer of the PS Cy5.5 cDNA1 was reduced by more than 80% following addition of the PS Iowa Black DNA1 even at the lowest 1:1 cDNA1:DNA1 molar ratio and more than 90% at cDNA1:DNA1 molar ratios 1:2 or higher. This quenching of Cy5.5 fluorescence was not apparent when the Iowa Black was added to the control nonspecific PS Cy5.5 rDNA. The figure presents results (N=3) obtained at 30 min and at the 5 M concentration of PS Cy5.5 cDNA 1.

The kinetics of quenching in wells (N=3) with 5 M PS Cy5.5 cDNA1 following addition of PS Iowa Black DNA1 at three cDNA1:DNA1 molar ratios from 1:1 to 1:3 showed that under the conditions of this in vitro study, quenching occurred extremely rapidly, reaching more than 80% within 5 min even at cDNA1:DNA1 molar ratio of 1:1 and greater than 90% at higher molar ratios. The quenching was persistent thereafter at least until the end of observation at 90 min. The quenching was found to be equally rapid in serum as well as in buffer (data not presented). Specifically compared to the fluorescence of wells without the Iowa Black, the fluorescence intensity in wells containing 5 M PS Cy5.5 cDNA1 in 75% serum fell by 73% immediately upon addition of the PS Iowa Black DNA1 at 1:1 molar ratio at room temperature and rose only slightly to 77% and 83% at 8 and 57 min, respectively. However, the quenching efficiency was somewhat reduced in serum over the 80% achieved in buffer under essentially identical experimental conditions.

FIG. 11B presents histograms showing that Cy5.5 on the 3′ end of the 36 mer PO cc'DNA even when tethered via MORF1 to streptavidin-coated microspheres loses 82% of its fluorescence by the addition of Iowa Black DNA2 in PBS/BSA buffer within 20 min at room-temperature even at a 1:1 molar ratio with little change at higher molar ratios or at 60 min (N=3).

### Animal Studies

Camera sensitivity was sufficient to provide an acceptable fluorescence image within 10 min following the administration of 0.3 nmol of PS Cy5.5 cDNA1 alone. However following administration of PS Iowa Black DNA1, image quality was markedly superior when 0.8 nmol of PS Cy5.5 cDNA1 was initially administered. Accordingly, the 0.8 nmol dosage of Cy5.5 was selected for subsequent studies.

Whole body ventral and dorsal fluorescent images of SKH-1 mice receiving either: nothing, PS Cy5.5 cDNA1 alone (0.8 nmol), and PS Cy5.5 cDNA1 followed by PS Iowa Black DNA1 at a 1 and 3 fold molar excess 10 min later and imaged 30 min thereafter, were analyzed to study the stoichiometry and kinetics of fluorescence hybridization quenching in vivo. Obvious quenching in both the ventral and dorsal views of PS Cy5.5 cDNA1 fluorescence following administration of PS Iowa Black DNA1 was revealed. As expected, the quenching was more pronounced following administration of PS Iowa Black DNA1 at a 3-fold molar excess. Fluorescence intensity in these hairless animals fed on a chlorophyll-free diet appeared to be concentrated in the kidneys as evident in the dorsal view and in the GI tract and possibly the thyroid as evident in the ventral view. The fluorescence from these and other tissues tended to change over time.

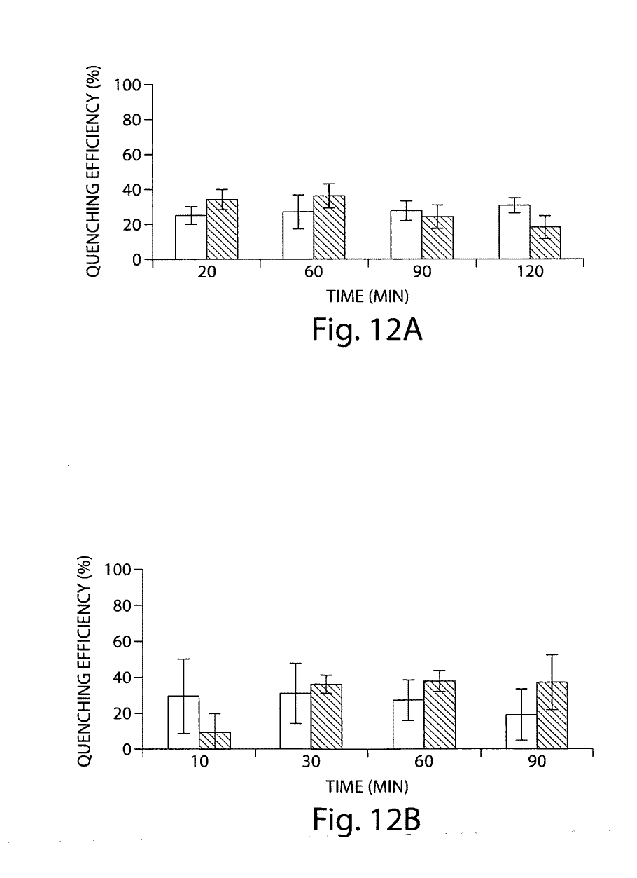

The quenching efficiency was determined by subtracting from unity the ratio obtained by dividing the whole body fluorescence intensity of the mice receiving both PS Cy5.5 cDNA1 and PS Iowa Black DNA1 by the fluorescence intensity of the mice receive PS Cy5.5 cDNA1 alone. As shown in FIG. 12A, when measured in this manner, the quenching efficiency at 20 min postadministration (N=3 in all cases) at a 3 fold molar excess of PS

Iowa Black DNA1 was 25.0±4.8% (dorsal view) and 34.1±5.6% (ventral view) with little change at 60 min (26.6±9.6% and 35.9±6.7%, respectively).

Thereafter, the streptavidin microspheres carrying 0.06 nmol of biotinylated MORF1/PO Cy5.5 cc′DNA were implanted intramuscularly in one thigh of SKH-1 mice and, after 15 min, each animal received either 0.06 nmol (N=2), 0.6 nmol (N=2), or 6.0 nmol (N=4) of the PS Iowa Black DNA2 by iv administration. One mouse received 6.0 nmol of the random PS Iowa Black rDNA2 as control. The animals were imaged in both the dorsal and ventral view immediately thereafter. The fluorescence intensity of the implanted thigh compared to that of the contralateral thigh was measured, and the decrease in this ratio after administration of the Iowa Black was defined as the percent quenching efficiency as before. FIG. 12B presents the results obtained over 90 min in mice receiving 6.0 nmol of the PS Iowa Black DNA2 at a 100-fold molar excess over the PO Cy5.5 (N=4). Lower dosage of Iowa Black DNA2 (10 and 1 fold molar excess) gave an unremarkable quenching effect. Specifically mice implanted with streptavidin beads binding the 36 mer PO Cy5.5 cc′DNA showed a decrease in target/nontarget ratio of 27%±12% (dorsal view) and 38%±6% (ventral view) within 1 h of administration of the 18 mer PS Iowa Black DNA2 but not the Iowa Black rDNA2 random control. When administered at a 100-fold molar excess, the target/nontarget ratio was increased 20% (dorsal view) and 14% (ventral view) at 40 min postadministration of the random Iowa Black rDNA2 control. Therefore, despite the barriers to in vivo hybridization including rapid clearance and poor access, the quenching of the tethered Cy5.5 fluorescence was remarkably effective following intravenous administration of the Iowa Black DNA2.

TABLE 3
Base Sequences of the PO and PS DNAs
and MORFf this Investigation^{a}

| oligomer | | |
|---|---|---|
| type | base sequence | modifications |
| MORF1, | 5′-TCT-TCT-ACT-TCA-CAA- | native or 3′ |
| 18 mer | CTA (SEQ ID NO: 5) | biotin |
| | | conjugated |
| PO DNA1, | 5′-TCT-TCT-ACT-TCA-CAA- | native only |
| 18 mer | CTA (SEQ ID NO: 5) | |
| PO cc′DNA | 5′-TAG-TTG-TGA-AGT-AGA- | native and 3′ |
| 36 mer | AGA-GGT-GTA-GGA-GTC- | Cy5.5 |
| | GGT-GTT (SEQ ID N0: 6) | conjugated |
| PS DNA1 | 5′-TCT-TCT-ACT-TCA-CAA- | native and 3′ |
| 18 mer | CTA (SEQ ID NO: 5) | Iowa Black |
| | | conjugated |
| PS cDNA1 | 5′-TAG-TTG-TGA-AGT-AGA- | 5′ Cy5.5 |
| 18 mer | AGA (SEQ ID NO: 7) | conjugated |
| PS random | 5′-AGA-ATG-ATG-GTT-ATA- | 15′ Cy5.5 |
| rDNA1 18 mer | AGG (SEQ ID NO: 8) | conjugated |
| PS random | 5′-ATA-CCA-ACC-GCC-TCC- | 5′ Iowa Black |
| rDNA2 18 mer | ACA (SEQ ID NO: 9) | conjugated |
| PS DNA2 | 5′-AAC-ACC-GAC-TCC-TAC- | 5′ Iowa Black |
| 18 mer | ACC (SEQ ID NO: 10) | conjugated |
| PS cc′DNA | 5′-TAG-TTG-TGA-AGT-AGA- | native |
| 36 mer | AGA-GGT-GTA-GGA-GTC- | |
| | GGT-GTT (SEQ ID N0: 6) | |

^{a}Complementary DNAs identified as cDNAs, random DNAs identified as rDNAs, DNAs with two complementary regions identified as cc′DNAs.

### Discussion

Pretargeting and similar applications of optical imaging have not been previously considered especially with linear oligomers. Recently an optical pretargeting study in which microspheres with a 25 mer cMORF were implanted in mice that subsequently received a duplex consisting of a 25 mer Cy5.5-MORF hybridized with a 18 mer BHQ3-PS DNA was completed (He, J. et al., Mol. Imaging. Biol. 9, 17-23, 2007). The results of that study confirm that fluorescent conjugated duplexes may be designed to dissociate rapidly in the presence of its target. Reported herein is an investigation of the in vivo stability of novel duplexes, the sensitivity of detection, the kinetics of hybridization and the pharmacokinetics of in vivo quenching. While these measurements are not directly related to pretargeting and similar applications of optical imaging, the results described herein will be required in the further development of optical imaging with linear duplexes.

Of the eight duplexes between PO DNA, PS DNA, and MORF, gel electrophoresis showed stability only for the MORF/PO DNA and possibly the MORF/PS DNA duplexes following incubations in 37° C. serum for 2 h and 4 h. To measure the kinetics of quenching, the fluorescence intensity of a Cy5.5 conjugated PS DNA was measured in vitro following addition of various molar ratios of Iowa Black conjugated complementary PS cDNA. The quenching was initially found to be essentially completed within 30 min (FIG. 11A) while a more focused study of kinetics indicated that quenching was completed within several minutes. By contrast no quenching was observed if the study Cy5.5 cDNA was replaced with the control Cy5.5 rDNA. Quenching in vivo in mice was also found to be rapid and essentially completed within 20 min (FIG. 12A).

The most plausible mechanism to explain the fluorescence inhibition of a Cy5.5 conjugated PS DNA following addition of the Iowa Black complementary but not random control DNA is contact fluorescence inhibition following duplex formation. That the quenching and therefore hybidization is rapid is encouraging since rapid kinetics will be required for the in vivo applications contemplated. Rapid hybridization was previously observed by surface plasmon resonance in which a 25 mer Cy5.5 MORF duplexed with an 18 mer BHQ3 DNA was allowed to flow over an immobilized 18 mer PS DNA complementary to the MORF (He, J. et al., Mol. Imaging. Biol. 9, 17-23, 2007). Dissociation of the duplex with capture of the MORF was completed in less than 1 min.

Whole body optical images of mice showed fluorescence inhibition at 30 min after the iv administration of Cy5.5-cDNA1 to mice administered iv Iowa Black-DNA1 10 min earlier. The results presented in FIG. 12A show that at 20 min post-administration of Iowa Black DNA1, the quenching efficiency was 25 to 34% depending upon the view, with little change thereafter.

Fluorescence was apparent at all dosages in hairless SKH-1 mice administered iv Cy5.5 cDNA1 alone; however, the administration of 0.8 nmol (5 g) of the Cy5.5 cDNA1 provided the most intense image. It can be concluded from these studies that the 0.8 nmol of Cy5.5 cDNA1 fluorescence may be detected with sufficient sensitivity and that the iv administration of 2.4 nmol of Iowa Black DNA1 (i.e., a 3 fold molar excess) provided obvious inhibition, superior to an equamolar administration and superior to the initial administration of 0.3 nmol of Cy5.5 cDNA1. The results confirm that a 0.8 nmol dosage of Cy5.5 cDNA1 provides sufficient fluorescence intensity for subsequent in vivo measurements with the camera used in this investigation, that fluorescence intensity is stable over at least 1 h, and that in vivo quenching of fluorescence occurs rapidly, consistent with the earliest practical times between administrations and imaging.

Having thus established that rapid fluorescence quenching follows hybridization in solution both in vitro and in vivo, it was necessary to ensure that immobilization does not adversely influence these properties. Since MORF/PO DNA duplexes are stable in serum, an 18 mer PO immobilized microsphere was constructed, consisting of a biotinylated MORF1 added first to the streptavidin-coated bead followed by addition of the 36 mer PO Cy5.5 DNA with two 18 mer complementary regions, one against the standard sequence (i.e., cDNA 1) and one against a new sequence (i.e., c′DNA2). Thereafter, the PS conjugated Iowa Black DNA2 quencher complementary to the 18 mer PO overhang of the bead duplex was administered, and the fluorescence intensity was measured. As shown in FIG. 11B, quenching inhibition in buffer was completed in 20 min or less and with about 82% inhibition at the lowest 1:1 molar ratio despite the immobilization.

Finally, as shown in FIG. 12B, in mice implanted with the 36 mer PO Cy5.5 cc′DNA microspheres showed a decrease in target/nontarget ratio of 27% (dorsal view) and 38% (ventral view) within 1 h of administration of the 18 mer PS Iowa Black DNA2 but not the Iowa Black rDNA2 random control. The quenching efficiency was therefore remarkable despite the rapid clearance from circulation of the PS Iowa Black DNA2 and the poor accessibility of the subcutaneous microspheres from the circulation.

In conclusion, the results of this proof of concept study show that duplexes of a fluorophore- and a quencher-conjugated linear oligomer can be designed to be sufficiently stable for use in vivo, that fluorescence quenching follows duplex hybridization of Cy 5.5 and Iowa Black conjugated oligomers and that this can be detected in mice at reasonable concentrations, that the hybridization and therefore quenching is rapid whether free or immobilized, and that the quenching is persistent. The results of this investigation of fluorescent quenching can be applied to applications such as optical antisense and pretargeting imaging.

What is claimed is:

1. A method of optically imaging a target entity in a subject, the method comprising
(a) administering to the subject a binding molecule that comprises an oligomer moiety and specifically binds to the target entity,
(b) contacting the binding molecule with a quenched first linear oligomer duplex comprising a detectably labeled oligomer, wherein the detectably labeled oligomer is hybridized to a quenching oligomer; the detectable label is quenched unless the detectably labeled oligomer and the quenching oligomer dissociate; and the detectably labeled oligomer has a higher affinity to form a linear duplex with the oligomer moiety of the binding molecule than to form a linear duplex with the quenching oligomer, and
(c) detecting the presence of unquenched detectable label in the subject, wherein the presence of unquenched detectable label indicates that the detectably labeled oligomer has dissociated from the quenching oligomer and hybridized to the oligomer of the binding molecule to form a second linear oligomer duplex, permitting optical imaging of the target entity in the subject.

2. The method of claim 1, wherein one or more of the oligomers are phosphodiester, phosphorothioate, peptide nucleic acid (PNA), locked nucleic acid (LNA), and/or phosphorodiamidate morpholino (MORF) oligomers.

3. The method of claim 1, wherein the detectable label is a fluorescent or bioluminescent label.

4. The method of claim 3, wherein the fluorescent label is a Cy5.5 emitter.

5. The method of claim 1, wherein the quenching oligomer comprises a quenching moiety.

6. The method of claim 5, wherein the quenching moiety is BHQ3 or Iowa black.

7. The method of claim 1, wherein the detectable label is detected in vivo.

8. The method of claim 1, wherein the detectable label is detected in real time.

9. The method of claim 1, wherein the first linear oligomer duplex comprises two oligomers that are not both phosphodiester, phosphorothioate, peptide nucleic acid (PNA), locked nucleic acid (LNA), or phosphorodiamidate morpholino (MORF) oligomers.

10. The method of claim 1, wherein the oligomer moiety is a single-stranded nucleic acid moiety.

11. The method of claim 1, wherein the binding molecule is an antibody or antigen-binding fragment thereof.

12. The method of claim 1, wherein the target entity is a polypeptide, nucleic acid, polysaccharide or lipid molecule.

13. The method of claim 1, wherein the target entity is a cell.

14. The method of claim 13, wherein the cell is a cancer cell.

15. The method of claim 1, wherein the subject is human.

16. The method of claim 1, wherein presence of the specific target entity is associated with a disease or disorder.

17. The method of claim 16, wherein the disease is cancer.

18. The method of claim 1, wherein the optical imaging of the target entity in the subject is diagnostic for a disease or disorder in the subject.

19. The method of claim 18, wherein the disease is cancer.

20. A method of optically imaging a target entity in a subject, the method comprising
(a) administering to the subject a linear oligomer duplex comprising a detectably labeled oligomer and a quenching oligomer, wherein the detectable label is quenched by the quenching oligomer unless the detectably labeled oligomer and quenching oligomer of the duplex dissociate, and wherein the detectably labeled oligomer has a higher affinity to form a duplex with a specific target nucleic acid than to form a duplex with the quenching oligomer, and
(b) detecting the presence of unquenched detectable label in the subject, wherein the presence of unquenched detectable label in the subject indicates that the detectably labeled oligomer has formed a duplex with the specific target nucleic acid, permitting optical imaging of the target nucleic acid in the subject.

21. The method of claim 20, wherein one or more of the oligomers are phosphodiester, phosphorothioate, peptide nucleic acid (PNA), locked nucleic acid (LNA), and/or phosphorodiamidate morpholino (MORF) oligomers.

22. The method of claim 20, wherein the detectable label is a fluorescent or bioluminescent label.

23. The method of claim 22, wherein the fluorescent label is a Cy5.5 emitter.

24. The method of claim 20, wherein the quenching oligomer comprises a quenching moiety.

25. The method of claim 24, wherein the quenching moiety is BHQ3 or Iowa black.

26. The method of claim 20, wherein the detectable label is detected in vivo.

27. The method of claim 20, wherein the detectable label is detected in real time.

28. The method of claim 20, wherein the specific target nucleic acid is an mRNA.

29. The method of claim 20, wherein the detectably labeled oligomer is an antisense sequence to the specific target nucleic acid.

30. The method of claim 20, wherein expression of the specific target nucleic acid is associated with a disease or disorder.

31. The method of claim 30, wherein the disease is cancer.

32. The method of claim 20, wherein the subject is human.

33. The method of claim 20, wherein the optical imaging of the target nucleic acid in the subject is diagnostic for a disease or disorder in the subject.

34. The method of claim 33, wherein the disease is cancer.

35. The method of claim 20, wherein the detectably labeled oligomer and the quenching oligomer are not both phosphodiester, phosphorothioate, peptide nucleic acid (PNA), locked nucleic acid (LNA), or phosphorodiamidate morpholino (MORF) oligomers.


SEQUENCE LISTING (10 sequences)

SEQ ID NO: 1 - DNA, 25 bp, Artificial Sequence
  Synthetic Oligonucleotide
tggtggtgggtgtacgtcacaacta

SEQ ID NO: 2 - DNA, 18 bp, Artificial Sequence
  Synthetic Oligonucleotide
tagttgtgacgtacaccc

SEQ ID NO: 3 - DNA, 25 bp, Artificial Sequence
  Synthetic Oligonucleotide
aagatccatcccgacctcgcgctcc

SEQ ID NO: 4 - DNA, 18 bp, Artificial Sequence
  Synthetic Oligonucleotide
ggagcgcgaggtcgggat

SEQ ID NO: 5 - DNA, 18 bp, Artificial Sequence
  Synthetic Oligonucleotide
tcttctacttcacaacta

SEQ ID NO: 6 - DNA, 36 bp, Artificial Sequence
  Synthetic Oligonucleotide
tagttgtgaagtagaagaggtgtaggagtcggtgtt

SEQ ID NO: 7 - DNA, 18 bp, Artificial Sequence
  Synthetic Oligonucleotide
tagttgtgaagtagaaga

SEQ ID NO: 8 - DNA, 18 bp, Artificial Sequence
  Synthetic Oligonucleotide
agaatgatggttataagg

SEQ ID NO: 9 - DNA, 18 bp, Artificial Sequence
  Synthetic Oligonucleotide
ataccaaccgcctccaca

SEQ ID NO: 10 - DNA, 18 bp, Artificial Sequence
  Synthetic Oligonucleotide
aacaccgactcctacacc